(19) United States
(12) Patent Application Publication
(10) Pub. No.: US 2017/0128491 A1
(43) Pub. Date: May 11, 2017

(54) CELL IMPLANT FOR ISLET GRAFTS AND USES THEREOF

(71) Applicant: Samsung Life Public Welfare Foundation, Seoul (KR)

(72) Inventors: Jae Hyeon Kim, Seoul (KR); Bae Jun OH, Incheon (KR); Sang Man Jin, Seoul (KR)

(21) Appl. No.: 15/266,772

(22) Filed: Sep. 15, 2016

(30) Foreign Application Priority Data
Sep. 15, 2015 (KR) 10-2015-0130441
Sep. 13, 2016 (KR) 10-2016-0117876

(51) Int. Cl. A61K 35/28; A61K 35/39

(52) U.S. Cl. CPC A61K 35/28; A61K 35/39; A61K 2035/124

(57) ABSTRACT

The present disclosure relates to a cell implant for islet grafts and uses thereof. More specifically, the present disclosure provides a method for treating or preventing diabetes including administering a composition comprising a spheroid and an islet cluster as active ingredients to a subject in need thereof, and a method for preparing the cell implant. According to the present disclosure, the cell implant can reduce loss of the biological activity and function of islet cells and increase the viability of the islet cells. Therefore, the cell implant can improve the quality of the islet cells or maintain the quality for a long time and also improve angiogenic capacity, and, thus, can be used in treating diabetes.

### CROSS-REFERENCE TO RELATED APPLICATIONS

This application is based on and claims priority from Korean Patent Application No. 10-2015-0130441, filed on Sep. 15, 2015 and No. 10-2016-0117876, filed on Sep. 13, 2016 with the Korean Intellectual Property Office, the disclosure of which is incorporated herein in its entirety by reference.

### TECHNICAL FIELD

The present disclosure relates to a cell implant for islet grafts and uses thereof.

### BACKGROUND

The number of patients suffering from diabetes is currently 4.6% of the world's population and expected to gradually increase and exceed 6.0% in 2025. Recently, pancreas transplantation and islet transplantation have been performed to treat diabetes. However, with a current technology of isolating and culturing cells for islet transplantation, it is possible to transplant islet cells obtained from two to four donors to only one diabetic patient, and maintenance of non-insulin dependency after transplantation does not exceed 10% (5 years). With introduction of immunosuppressant, the outcome of transplantation has been continuously improved, but a dramatic breakthrough in clinical islet transplantation for diabetes treatment is still needed.

Accordingly, studies for improving the viability of implant by optimizing islet isolation and culturing conditions and minimizing damage to the transplanted islet are being conducted. Also, various methods for proliferating islet cells and xenotransplantation methods using non-human animal tissues are being studied.

Since the islet transplantation protocol was established in Edmonton, Canada, clinical islet transplantation has been regarded as a treatment method for treating type 1 diabetics. However, the low engraftment of transplanted islet cells becomes a major cause of failure of long-term blood sugar regulation. It is necessary for islet cells to be successfully engrafted through revascularization and blood flow regulation within a few days after transplantation. However, the transplanted islet cells are exposed to a state with low vascular density and oxygen partial pressure and low-nutrient condition as compared with endogenous islet cells, and thus many cells die. Therefore, it is difficult to achieve normal engraftment of islet cells and there may be a problem in regulating insulin secretion.

Further, pancreas islet cells form blood vessels. Thus, formation of blood vessels damaged during islet transplantation is one of the most important factors contributing to destruction of islet cells during an initial transplantation period. However, a new blood-vessel network can be completed about 10 to 14 days after transplantation.

Meanwhile, it has been reported that as compared with a monolayer cell culture method, a spheroid culture method enables mass-proliferation of stem cells by cell-cell contact and cell-matrix contact and shows angiogenic capacity against ischemic diseases due to paracrine effects caused by various proangiogenic factors.

Therefore, in order to finally treat diabetes, an efficient strategy for increasing autologous treatment cell groups capable of improving the quality of islet cells before transplantation or maintaining the quality for a long time and also having a high angiogenic capacity with paracrine effects is needed.

### SUMMARY

The inventors of the present disclosure have made intensive efforts to develop a medicine for diabetes. As a result, the inventors of the present disclosure have found that angiogenesis can be promoted and engraftment of islet can be increased by co-transplanting a bone marrow-derived angiogenic spheroid and an islet cluster and thus completed the present disclosure.

Accordingly, it is an object of this invention to provide a cell implant for islet transplantation.

It is another object of this invention to provide a pharmaceutical composition for treating or preventing diabetes.

It is still another object of this invention to provide a method of for preparing a cell implant for islet transplantation.

Hereinafter, the present disclosure will be described in more detail.

In one aspect of the present invention, there is provided a cell implant for islet transplantation comprising a spheroid and an islet cluster as active ingredients.

According to an exemplary embodiment of the present disclosure, the spheroid is derived from bone marrow mononuclear cells.

In order to prepare the cell implant for islet transplantation according to the present disclosure, a bone marrow mononuclear cell-derived spheroid and an islet cluster are separately prepared.

Isolation and culturing of pancreatic cells from pancreatic tissues for preparing the islet cluster can be performed using various methods known in the art.

Isolation and culturing of bone marrow and bone marrow mononuclear cells for preparing the spheroid can be performed using various methods known in the art.

The bone marrow can be obtained from a mammal. The mammal may be selected from the group consisting of human, cow, horse, pig, goat, dog, cat, chicken, mouse, rat, rabbit, and guinea pig.

The bone marrow mononuclear cells of the present disclosure refer to not only mesenchymal stromal cells, but also a heterogeneous population including hematopoietic cell lines such as lymphocytes, monocytes, stem cells, and precursor cells. In the present disclosure, bone marrow mononuclear cells isolated from bone marrow by density gradient isolation are used.

The stem cells may include hematopoietic stem cells (HSCs) and endothelial progenitor cells (EPCs), and refer to cells which can be differentiated into specific cells under specific conditions.

The term “bone marrow mononuclear cell-derived spheroid”, “bone marrow-derived spheroid(BM-spheroid)”, or “spheroid” used herein means an aggregate of parenchymal culture cells derived from bone marrow mononuclear cells and having a substantially spherical shape, and may have a shape as shown in a micrograph of FIG. 1A or a shape similar thereto.

The term “substantially spherical shape” is not limited to a completely spherical shape, but may include a somewhat flattened spherical shape.

According to an example of the present disclosure, the spheroid may mean a cell obtained by isolating bone marrow from the femoral region and the tibia of a mammal, obtaining a mononuclear cell by centrifugation of the bone marrow, and then culturing the mononuclear cell in a EGM-2 medium on an ultra-low attach surface for 5 days.

According to an exemplary embodiment of the present disclosure, the spheroid is co-transplanted with the islet cluster through the renal capsule or hepatic portal vein.

One of the main features of the present disclosure is that a spheroid and an islet cluster are individually and separately prepared to prepare a cell implant for islet transplantation and then co-transplanted by individually and separately injecting them or by injecting a mixture of the spheroid and the islet cluster.

Further, in order for the spheroid and the islet cluster injected into the body to stably reach an actual target site and show a required treatment effect, various requirements need to be met. Firstly, if spheroids are in a suitable size when being injected into the hepatic portal vein, the cells injected into the hepatic portal vein may suppress reduction of a blood flow rate or thrombopoiesis.

For example, if a spheroid having a relatively large size is injected into the body, it may affect the activity in blood vessels. Specifically, it may reduce a blood flow rate and may disturb blood circulation and thus result in stoppage of blood flow, thrombopoiesis, angiostenosis, and even death. On the contrary, if a spheroid having a small size is injected, the spheroid may not be engrafted due to a blood flow.

Therefore, it is desirable to inject a spheroid islet cluster having a suitable size into the hepatic portal vein. Further, preferably, cells may not be broken or aggregated before being injected into a blood vessel and even after being injected, and then may stably reach a target site without the broken and aggregated cells. Further, in order for the cells reaching the target site to show a required treatment effect, it is desirable to inject cells with a predetermined concentration or more. The size of the spheroid of the present disclosure from among the various requirements is similar to the size of an islet and thus suitable for injection into the body and engraftment. Therefore, the spheroid injected into a blood vessel stably shows a treatment effect without reducing a blood flow rate or forming thrombi.

The term “suitable size” used herein when describing a spheroid means a size which can be advantageous to a spheroid injected into the body (e.g., hepatic portal vein) in readily moving to a target tissue without disturbing blood flow or circulation, and being engrafted into the target tissue and then showing the activity and efficacy in the target tissue.

Since the cell implant of the present disclosure needs to be injected into the body through a specific route, e.g., a blood vessel such as hepatic portal vein, preferably, the spheroid of the present disclosure may have a suitable size for injection into the body. Further, most preferably, the spheroid may have a size identical or similar to the size of an islet cell to be transplanted.

The spheroid having the suitable size identical or similar to the size of the islet cluster is not limited as long as the object of the present disclosure can be achieved. However, in the suitable size of the spheroid of the present disclosure, a diameter of the spheroid may be different by 0 to 500 μm or less, preferably 0 to 200 μm or less and most preferably 0 to 50 μm or less, from a diameter of the islet cluster.

According to an exemplary embodiment of the present disclosure, the spheroid may have the suitable size identical or similar to the size of the islet cluster. A diameter of the spheroid may be in the range of from 10 to 500 μm, and more preferably in the range of from 50 to 350 μm.

In the present disclosure, the spheroid has a size similar to the size of the islet cluster. By transplanting the spheroid, it is possible for the spheroid to remain in the liver for a longer time and thus possible to improve the function of islet cells.

According to an exemplary embodiment of the present disclosure, in the spheroid, CD14, CD34, CXCR4, CD14/CXCR4, or CD14/CD34 is overexpressed, as compared with a non-spheroid.

Further, the spheroid of the present disclosure has the characteristics of a mononuclear cell and a hematopoietic stem cell. According to an example of the present disclosure, in the spheroid of the present disclosure, CXCR4 and CD14 as monocyte markers and CD34 as a hematopoietic stem cell marker are overexpressed.

The term “overexpression” used herein may refer to a case where a gene and/or protein is analyzed as being highly expressed as compared with a control into which the spheroid is not transplanted when gene expression or protein expression is analyzed according to a molecular biological experiment by gene expression analysis methods, such as RT-PCR (see Sambrook, J. et al., Molecular Cloning. A Laboratory Manual, 3rd ed. Cold Spring Harbor Press (2001)), Western Blotting (“Imaging Systems for Westerns: Chemiluminescence vs. Infrared Detection, 2009, Methods in Molecular Biology, Protein Blotting and Detection, vol. 536”. Humana Press. Retrieved 2010), FACS (Flow cytometry or Fluorescence-activated cell sorting, Loken M R (1990). Immunofluorescence Techniques in Flow Cytometry and Sorting (2nd ed.). Wiley. pp. 34153), and Immunocytochemistry Methods and Protocols (edited by Lorette C. Javois, 2nd edition, 1999. Human Press) typically used in the art.

The spheroid of the present disclosure may have a DiI-ac-LDL (Acetylated Low Density Lipoprotein labeled with 1,1′-dioctadecyl-3,3,3′,3′-tetramethylindo-carbocyanine perchlorate) absorbing capacity and a BS-1 (Bandeiraea simplicifolia-derived) lectin binding capacity.

The DiI-ac-LDL absorbing capacity is a marker for confirming a spheroid. If the cell is in contact with DiI-ac-LDL, lipoprotein is broken down by a lysosomal enzyme and DiI, which is a fluorescent dye, is accumulated in an endomembrane so as to be visually seen.

The BS-1 lectin binding capacity is a marker for confirming contribution of a spheroid to angiogenesis. If a spheroid reacts with BS-1 lectin, the spheroid may have a capacity of binding BS-1.

According to an exemplary embodiment of the present disclosure, the spheroid and the islet cluster may include cells derived from autologous, allogenic, or xenogeneic tissues or two or more kinds of cells derived therefrom. Most preferably, the spheroid and the islet cluster may be autologous.

If the bone marrow-derived spheroid and the islet cluster forming the cell implant of the present disclosure are co-transplanted, the viability is increased as compared with a control. Further, the angiogenic capacity is promoted with an excellent capacity of regulating a normal blood sugar level.

That is, in the cell implant of the present disclosure, the bone marrow-derived spheroid transplanted together with the islet cluster may affect settle stabilization of initial islet cells and thus improve the functions (glucose tolerance test and insulin secretion) of islet cells.

Further, in the cell implant of the present disclosure, the bone marrow-derived spheroid transplanted together with the islet cluster may improve proliferation of beta cells and contribute to revascularization in an incorporation manner to form or newly form a blood vessel. Thus, it can be applied to prevention and treatment of various ischemic diseases such as diabetes.

The term “blood sugar regulation capacity” used herein refers to a capacity of a cell implant to reach a normal blood sugar level when the cell implant is transplanted into a mouse. More specifically, it can be confirmed on the basis of a cumulative percentage of normal blood sugar level arrival with respect to the cell implant and an improved change in blood sugar level after intraperitoneal glucose tolerance tests (IPGTT).

The term “angiogenic capacity” used herein refers to a vascularization capacity of the cell implant. Specifically, the degree of angiogenesis by the cell implant can be confirmed by performing immunocytochemistry using CD31 antibody (on the basis of a mouse) and counting the number of generated blood vessels.

In another aspect of the present invention, there is provided a pharmaceutical composition for treating or preventing diabetes, the pharmaceutical composition comprising the above-described cell implant as an active ingredient.

According to an exemplary embodiment of the present disclosure, the diabetes may be type 1 diabetes.

If the composition of the present disclosure is prepared as a pharmaceutical composition, the pharmaceutical composition of the present disclosure may include a pharmaceutically acceptable carrier. The pharmaceutically acceptable carrier included in the pharmaceutical composition of the present disclosure is one commonly used in formulations and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., but is not limited thereto. The pharmaceutical composition of the present disclosure may further include a lubricant, a wetting agent, a sweetener, a flavor, an emulsifier, a suspending agent, a preservative, or the like in addition to the above-described ingredients. Suitable pharmaceutically acceptable carriers and formulations are described in detail in Remington's Pharmaceutical Sciences (19th ed., 1995).

The pharmaceutical composition of the present disclosure may be administered parenterally. In case of parenteral administration, it may be administered through local injection.

An adequate dose of the pharmaceutical composition of the present disclosure may be determined according to various factors including methods of formulation, administration modes, ages, weights, sex, pathological conditions and diet of patients, administration periods, administration routes, excretion rates and reaction sensitivity. Preferably, a dose of the pharmaceutical composition of the present disclosure for an adult may be 1 to 10000 cells/kg (body weight).

The pharmaceutical composition of the present disclosure may be prepared according to a method that may be easily carried out by those skilled in the art in single-dose forms or in multi-dose packages using a pharmaceutically acceptable carrier and/or excipient. In this case, a formulation of the composition may be solution in an oil or aqueous medium, suspension, syrup, emulsion, extract, discutient, powder, granule, tablet, or capsule, and may further include a dispersant or a stabilizer.

The composition for preventing or treating diabetes of the present disclosure includes the above-described cell implant as an active ingredient. Thus, overlapping descriptions between the two embodiments will be omitted in order to avoid excessive complexity of the present specification.

In still another aspect of this invention, there is provided a method for treating or preventing diabetes, comprising administering the composition comprising a spheroid and an islet cluster as active ingredients to a subject in need thereof.

The method for treating or preventing diabetes of the present disclosure includes the above-described cell implant or composition as an active ingredient. Thus, overlapping descriptions between the two embodiments will be omitted in order to avoid excessive complexity of the present specification.

In further aspect of this invention, there is provided a method for preparing a cell implant for islet transplantation, including the following steps:

(a) preparing a spheroid derived from bone marrow mononuclear cells; and

(b) preparing a cell implant by mixing the spheroid and an islet cluster.

According to an exemplary embodiment of the present disclosure, in the cell implant of the step (b), a cell mixing ratio of the spheroid and the islet cluster is 1000:1 to 10000:1.

That is, according to the present disclosure, it is possible to prepare a cell implant which can be readily moved to a target tissue and has a high stability. Thus, it is possible to dramatically improve the efficacy of treating cells by administration of the cell implant.

The method according to the present disclosure is a method for preparing the above-described cell implant. Thus, overlapping descriptions between the two embodiments will be omitted in order to avoid excessive complexity of the present specification.

According to the exemplary embodiments of the present disclosure, a cell implant of the present disclosure includes a spheroid and thus reduces loss of the biological activity and function of islet cells and increases the viability of the islet cells. Therefore, the cell implant can improve the quality of the islet cells or maintain the quality for a long time and also improve angiogenic capacity, and, thus, can be used in treating diabetes.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows immunostaining images of mouse bone marrow-derived (BM) spheroids generated on an ultra-low attach surface using three-dimensional culture.

FIG. 1B shows a chart of the diameter of spheroids versus culture period days.

FIG. 1C shows the results of a flow cytometry analysis on CD14, CD34, CXCR4 and CD31 protein expression.

FIG. 2A shows staining images of cells stained with DiI (positive population) and cells not stained with DiI (negative population).

FIG. 2B shows images of positive population and negative population.

FIG. 2C shows confocal microscope images of the inside of the spheroid.

FIG. 3A shows images of tube structures that came out of the bone marrow-derived spheroids.

FIG. 3B shows immunostaining images of tube structures.

FIG. 3C shows images of incorporation levels of GFP-BM spheroids, fresh bone marrow mononuclear cells and BM-non-spheroids into blood vessel structures.

FIG. 3D shows images angiogenic capacity of GFP-BM spheroids, fresh bone marrow mononuclear cells and BM-non-spheroids.

FIG. 3E shows several charts of relative tube length and mRNA expression of the factors involved in proliferation of blood vessels.

FIG. 4A shows images of angiogenic capacity of BM spheroids, fresh bone marrow mononuclear cells and BM-non-spheroids.

FIG. 4B shows a chart of vessel numbers of BM spheroids, fresh bone marrow mononuclear cells and BM-non-spheroids.

FIG. 4C shows images of GFP-BM-spheroids.

FIG. 5A shows a chart of blood glucose levels versus time after transplantation of islet cluster and spheroids into syngeneic diabetes mouse model.

FIG. 5B shows a chart of normoglycerimia versus time in the same model.

FIG. 5C shows a chart of blood glucose levels versus time after glucose injection in the same model.

FIG. 5D shows a bar graph of Area Under the glucose Curve in the same model.

FIG. 5E shows a bar graph of insulin in the same model

FIG. 6A shows immunofluorescent staining images of BM-spheroid and islet cluster co-transplanted cells.

FIG. 6B shows a bar graph of vessel numbers in the same cells

FIG. 6C shows a bar graph of the size of the total graft sections (endocrine and non-endocrine) in the same cells.

FIG. 6D shows a bar graph of glucagon-positive cells in the same cells.

FIG. 6E shows images of the same cells demonstrating improved proliferation of beta cells.

FIG. 6F shows a bar graph of Ki 67/insulin and nuclei in the same cells.

FIG. 7A shows images of stained blood vessels in the cells injected with lectin.

FIG. 7B shows a bar graph of blood vessel numbers in the same cells.

FIG. 7C shows a bar graph of incorporated GFP+ cells in the same cells.

FIG. 7D shows images of the same cells demonstrating distribution of the transplanted GFP-spheroid.

FIG. 8 shows the composition of human islet cells according to size.

FIG. 9A shows a photograph of a single cell and BM-spheroids.

FIG. 9B shows a chart of blood glucose levels versus time after transplantation.

FIG. 9C shows another chart of blood glucose levels versus time after glucose injection (intraperitoneal glucose tolerance test).

FIG. 10 shows the result of comparison about effects between a BM-spheroid of the present disclosure and a mesenchymal stem cell (MSC)-derived spheroid.

FIG. 11 is a schematic diagram showing comparison depending on the degree of integration of spheroids.

FIG. 12A shows a graph of blood glucose levels in islets alone, islets plus spheroids and islets plus dissociated spheroids.

FIG. 12B shows a chart of normoglycemia versus days after transplantation in the same cells.

FIG. 13A shows a chart of blood glucose levels in diabetic mice transplanted with islet alone, islets plus BM-spheroids, and islets plus dissociated-spheroids.

FIG. 13B shows a bar graph of AUC glucose levels in the same model.

FIG. 13C shows a chart of serum insulin levels in the same model.

FIG. 14A shows morphologic images of transplanted cells which are islets alone, islets plus BM-spheroids, and islets pluse dissociated-spheroids.

FIG. 14B shows a bar graph of franctional beta cell area in the same cells.

FIG. 14C shows a bar graph of islet size in the same cells.

FIG. 14D shows a bar graph of islet numbers per total area.

FIG. 15A shows images of islets alone, islets plus spheroids and islets plus dissociated spheroids transplanted to diabetic mice.

FIG. 15B shows a bar chart of vascular density in the same model.

FIG. 16A shows an in vitro MRI image of BM-spheroids.

FIG. 16B shows in vitro MRI images of normal cells, BM-spheroid, and BM-non-spheroid.

FIG. 16C shows ex-vivo MRI images of the same cells.

FIG. 16D shows optical imaging of GFP-BM spheroids.

FIG. 17A shows a schematic diagram of transplantation of spheroids.

FIG. 17B shows a chart of blood glucose levels in islet alone cells.

FIG. 17C shows a chart of blood glucose levels in islet plus spheroid cells.

FIG. 17D shows a chart of blood glucose levels in islet plus dissociated-spheroid cells.

FIG. 17E shows a chart of normoglycemia versus days after transplantation in the same cells.

FIG. 18A shows morphologic images of transplanted cells which are islets alone, islets plus BM-spheroids, and islets plus dissociated-spheroids.

FIG. 18B shows bar graphs of fractional beta cell area, islet size and islet number per total area in the same cells.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

In the following, the examples will be provided only for illustrating the present disclosure in more detail. According to the gist of the present disclosure, it will be obvious to those skilled in the art that the scope of the present disclosure is not limited to these examples.

### Experimental Materials and Methods

Isolation of Mononuclear Cell from Mouse Bone Marrow and Preparation of Spheroid

Mononuclear cells were isolated from mouse bone marrow to prepare spheroids as follows.

Bone marrow (BM) was collected from the femoral region and the tibia of normal C57BL/6J mice and green fluorescent protein-transgenic mice (GFP-Tg) derived from 10 to 12 weeks male C57BL/6J. After muscles and connective tissues were removed from bones, 1×PBS was injected using a 30-gauge injector to obtain bone marrow in the bones. Then, mononuclear cells (MNCs) were isolated from the bone marrow by density gradient centrifugation using Histopaque-1083. Bone marrow cells isolated from the GFP-Tg were used to track transplanted cells.

In order to prepare bone marrow-derived spheroids (BM-spheroids) from the bone marrow mononuclear cells(MNCs), the bone marrow MNCs were mixed in an EGM-2+5% FBS culture medium at a density of 3 to 5×10⁶ cells/ml and then inoculated into a HydroCell™ ultra-low attach surface dish. After 2-day culture, a fresh culture medium was added thereto. Then, the cells were further cultured for 3 days. The prepared spheroids were used for in vitro and in vivo experiments.

### Isolation of Islet Cluster

0.8 mg/kg collagenase P was injected into the pancreas of a mouse through the common bile duct to isolate a cluster as the aggregation of islet cells. The cluster was purified using Ficoll density gradient. The islet cells were cultured in RPMI 1640 containing 10% FBS and 1% penicillin/streptomycin. Next day, only the islet cluster was sorted with a pipette under a dissecting microscope and then used for transplantation.

### Flow Cytometry Analysis

A flow cytometry analysis was carried out as follows to check the composition of fresh bone marrow mononuclear cells (BM-MNCs), bone marrow-derived spheroids (BM-spheroids), and bone marrow non-spheroids (BM-non-spheroids) (simply isolated bone marrow mononuclear cells).

The BM-spheroids were treated with dispase (100 μg/ml) to dissociate them into individual cells. The cells (1 to 2×10⁵) were put in 100 μl of a buffer added with 0.5% BSA with various concentrations of antibodies and then stored at 4° C. for 20 minutes. Then, the cells were washed with 1×PBS twice, and a flow cytometry analysis was carried out. As the antibodies, PE (phycoerythrin)- or FITC (fluorescein isothiocyanate)-conjugated anti-mouse CD31, PE-anti-mouse CD14, FITC-anti-mouse CD45, PE-anti-mouse CXCR4 or APC-anti-mouse CXCR4, PE-Cy5-CD3 and FITC or eFluor 660-conjugated anti-mouse CD34 were used. The analysis was carried out using CellQuestPro software. In order to evaluate viabilities of the BM-non-spheroids and the BM-spheroids, Calcein-AM and PI (Propidium Iodide) were used. Each cell was treated with 2 μM Calcein-AM and stored at 37° C. for 25 minutes and then washed. Then, each cell was treated with 2.5 μM PI and a flow cytometry analysis was carried out.

### Angiogenesis Test on Matrigel

In order to find out whether or not BM-spheroids incorporate into a tube structure formed by human umbilical vein endothelial cells (HUVECs), 250 μl matrigel was coated on a 24-well plate and kept at 37° C. for 30 minutes. In order to track BM-spheroids incorporating into a tube structure formed by HUVECs, bone marrow derived from GFP was used and HUVECs were cultured on the matrigel together with fresh BM-MNCs, BM-spheroids, BM-non-spheroids, and BM-dissociated spheroids dissociated into individual cells.

Further, indirect effects of co-culture were examined. In order to isolate the HUVECs cultured on the matrigel, the fresh BM-MNCs, the BM-spheroids, and the BM-non-spheroids were placed on a culture membrane insert well and co-cultured. The cells were cultured in EBM-2 added with 1% FBS. After 24 hours, tube structures formed by the HUVECs cultured together with the respective cells were compared, and cells of the tube structures were labelled with 25 μg/ml Calcein-AM and the tubes labelled with a fluorescent dye were observed under a fluorescence microscope.

### In Vivo Matrigel Plug Assay

An in vivo matrigel plug assay was carried out to observe blood vessels newly generated in a gel plug transplanted together with cells under the skin of a mouse. In order to evaluate the degree of contribution of cells transplanted in vivo to angiogenesis, 1×10⁶ GFP-BM-spheroids, BM-non-spheroids, or fresh BM-MNCs were used, and the cells were mixed in 300 μl matrigel and then subcutaneously injected into the flank of a mouse. After 14 days, the transplanted matrigel mass was collected and fixed to 4% PFA for fluorescent staining. The tissue was kept in a freezer using an OCT compound. The tissue was cut into 10 μm pieces. As for the newly generated blood vessels, the degree of angiogenesis for each cell group was compared using CD31 antibody.

Isolation of CD31+, CD14+, and CD34+ from Bone Marrow Mononuclear Cell

In order to find out which cell group incorporates in formation of spheroids, CD31, CD14, and CD34 positive cells were isolated from BM-MNCs through flow cytometry analysis using PE-CD31, PE-CD14, and APC-CD34 antibodies. The cells were put in 100 μl of a buffer added with 0.5% BSA with various concentrations of each of the antibodies and then stored at 4° C. for 20 minutes. Then, the cells were washed with 1×PBS twice and isolated using FACS Aria III. The isolated positive cells for each antibody were stained with CM-DiI (1 μm). Then, the positive cells stained with DiI were co-cultured with negative cells respectively corresponding thereto in a spheroid culture medium for 5 days. In order to confirm whether the positive cells stained with DiI are present in the middle of the spheroids, GFP-CD31, CD14, and CD34 positive cells were obtained in the same manner as described above from GFP-mononuclear cells obtained from GFP-Tg mice and negative cells were obtained from normal mice and then co-cultured for 5 days. Then, a Zeiss CLSM780 confocal microscope was used to confirm whether GFP-positive cells are distributed in the middle of the spheroids.

### Co-Transplantation of BM-Spheroid and Islet Cluster and Result Evaluation of Co-Transplantation

A 10-12 week GFP-Tg mouse and a normal mouse were used as a donor and a recipient depending on experimental conditions. The GFP-Tg mouse was used to evaluate the degree of contribution of blood vessels derived from a donor. A diabetes mouse model was intraperitoneally injected with 180 mg/kg streptozotocin (STZ). A blood sugar measurement was carried out by collecting blood from the tail vein and using a glucose meter. Only a mouse with a constantly high blood sugar level (=20 mmol/1) was used for transplantation. An islet cluster and BM-spheroids were transplanted into the left renal capsule or the hepatic portal vein of a syngeneic recipient. For the co-transplantation experiment, 200 islet clusters and 240 BM-spheroids (each spheroid includes 4.2±1.3×10³ mononuclear cells and the total number of cells is 1×10⁶), or 200 islet clusters and 1×10⁶ BM-non-spheroids were used for transplantation. After transplantation, a body weight and a blood sugar level were measured twice per week. The kidney including a transplantation site was removed on the 14^{th} day and on the 28^{th} day, and then immunohistochemical staining and morphological analysis were carried out. In order to evaluate functional vessels, 200 μg TRITC-BS (tetramethyl rhodamine isothiocyanate-bandeiraea simplicifolia)1-lectin was injected through the tail vein before the kidney was removed, and after 1 hour, the transplantation site was obtained. On the 28^{th} day, intraperitoneal glucose tolerance tests (IPGTT) were conducted. After fasting for 16 hours, 1 g/kg sugar was intraperitoneally injected and a blood sugar level was measured 0, 15, 30, 45, 60, 90, and 120 minutes after injection. At 0 and 30 minutes, blood was obtained through a capillary vessel from the side of eyeball. The isolated serum insulin was measured using a rat/mouse insulin enzyme-linked immunosorbent assay (ELISA) kit. The percentage of mice reaching the normal blood sugar level and the reaching time were calculated for each group. When a blood sugar level was reduced by <11/1 mmol/1 for two consecutive days during the blood sugar test, it was determined as being effective.

### Immunostaining

On the 28^{th} day after transplantation, the kidney was extracted and fixed in 4% paraformaldehyde and washed with PBS and then immersed in 30% sucrose to prepare a frozen block. The tissue was cut into 10 μm pieces to be stained. The tissues were stained using an insulin antibody for staining islet cells, a CD31 antibody for staining new blood vessels, Ki67 and BrdU antibodies for confirming proliferation of beta cells, a GFP antibody for staining GFP-derived cells, and a glucagon antibody for staining alpha cells. The tissues were blocked with 10% normal goat serum. Then, rat anti-mouse CD31, rabbit polyclonal anti-GFP, rabbit anti-Ki67, -BrdU, guinea pig anti-insulin, and rabbit anti-glucagon antibodies were used as primary antibodies, and 568-conjugated goat anti-rat, 488 or 568-conjugated goat anti-rabbit, and Cy3-conjugated anti-guinea pig were used as secondary antibodies. DAPI was used for nuclear staining Morphological measurement and analysis were conducted using Image-Pro Plus software version 5.1.

### Statistical Analysis

The data were indicated as mean values±standard errors. Differences between groups were analyzed by a two-tailed unpaired t-test, a log-rank test, and a one-way analysis of variance (ANOVA), and a p-value of less than 0.05 was regarded as being statistically significant.

### Example 1. Preparation of Bone Marrow-Derived Spheroid Using Three-Dimensional Culture

The inventors of the present disclosure cultured mouse bone marrow mononuclear cells by a three-dimensional culturing method.

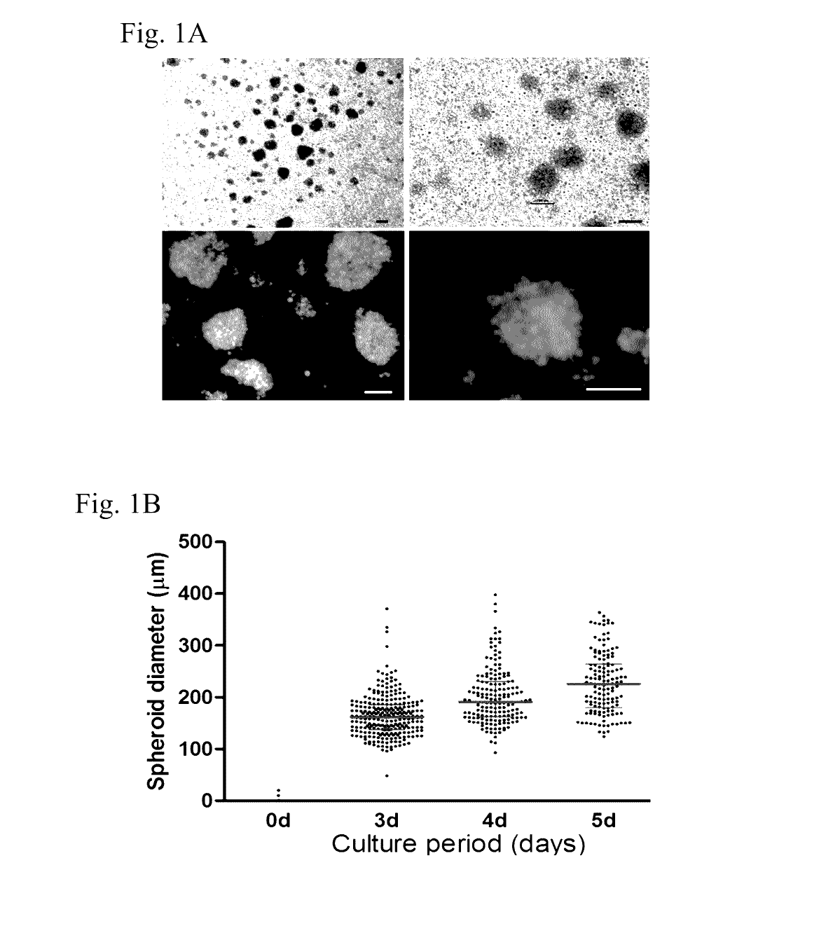

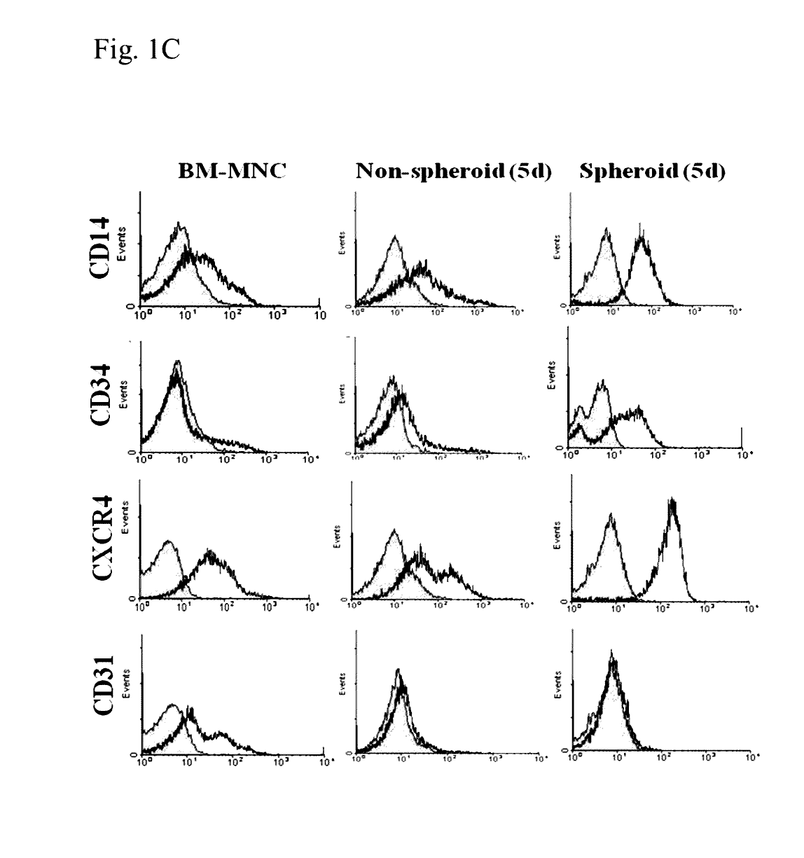

As a result, BM-spheroids were naturally generated on an ultra-low attach surface, and the shape and size of the spheroids were similar to those of the previously reported result (FIGS. 1A and 1B).

Further, in order to compare surface protein markers of a BM-spheroid generated on the 5^{th} day and a fresh bone marrow mononuclear cell, a flow cytometry analysis on CD14, CD34, CXCR4, and CD31 was conducted.

As a result, the BM-spheroid had a higher expression level than the fresh bone marrow mononuclear cell and a BM-non-spheroid generated on the 5^{th} day as shown in in FIG. 1C. Although the BM-spheroid mostly expressed CD14, an expression level of CD31 was lower than that of the fresh bone marrow mononuclear cell. Further, the BM-spheroid showed significantly higher expression levels of CD14+/CXCR4+ and CD14+/CD34+ than the fresh bone marrow mononuclear cell and the BM-non-spheroid (Table 1).

TABLE 1
Cell surface-based characterization of fresh BM-MNCs, BM-spheroids and BM-nonspheroids by FACS analysis

| | Culture | | | | | | | |
|---|---|---|---|---|---|---|---|---|
| Cell Type | (days) | CD14⁺ | CD31⁺ | CD34⁺ | CXCR4⁺ | CD31⁺/CXCR4⁺ | CD14⁺/CXCR4⁺ | CD14⁺/CD34⁺ |
| BM-MNCs | 0 | 29.2 ± 6.9 | 24.8 ± 6.4 | 16.7 ± 2.0 | 87.0 ± 4.3 | 24.4 ± 3.6 | 29.5 ± 3.2 | 13.2 ± 2.3 |
| BM- | 5 | 25.8 ± 2.9 | 2.9 ± 0.9 | 5.3 ± 1.7 | 34.7 ± 3.8 | 1.4 ± 0.4 | 21.2 ± 2.2 | 5.0 ± 1.1 |
| nonspheriod | | | | | | | | |
| BM- | 5 | 77.8 ± 7.2* | 2.0 ± 0.2 | 47.8 ± 2.0* | 81.3 ± 8.1** | 2.1 ± 0.3 | 72.8 ± 6.6* | 41.2 ± 1.8* |
| spheroid | | | | | | | | |

All experiments are performed in triplicate (n = 3). Values are indicated as mean ± SEM (%).
*P <0.05 versus BM-MNCs or BM-nonspheroid.
**P <0.05 versus BM-nonspheroid

### Example 2. Confirmation of Blast Group Forming Mouse Bone Marrow-Derived Spheroid

The inventors of the present disclosure observed a blast group forming the mouse bone marrow-derived spheroid prepared in Example 1.

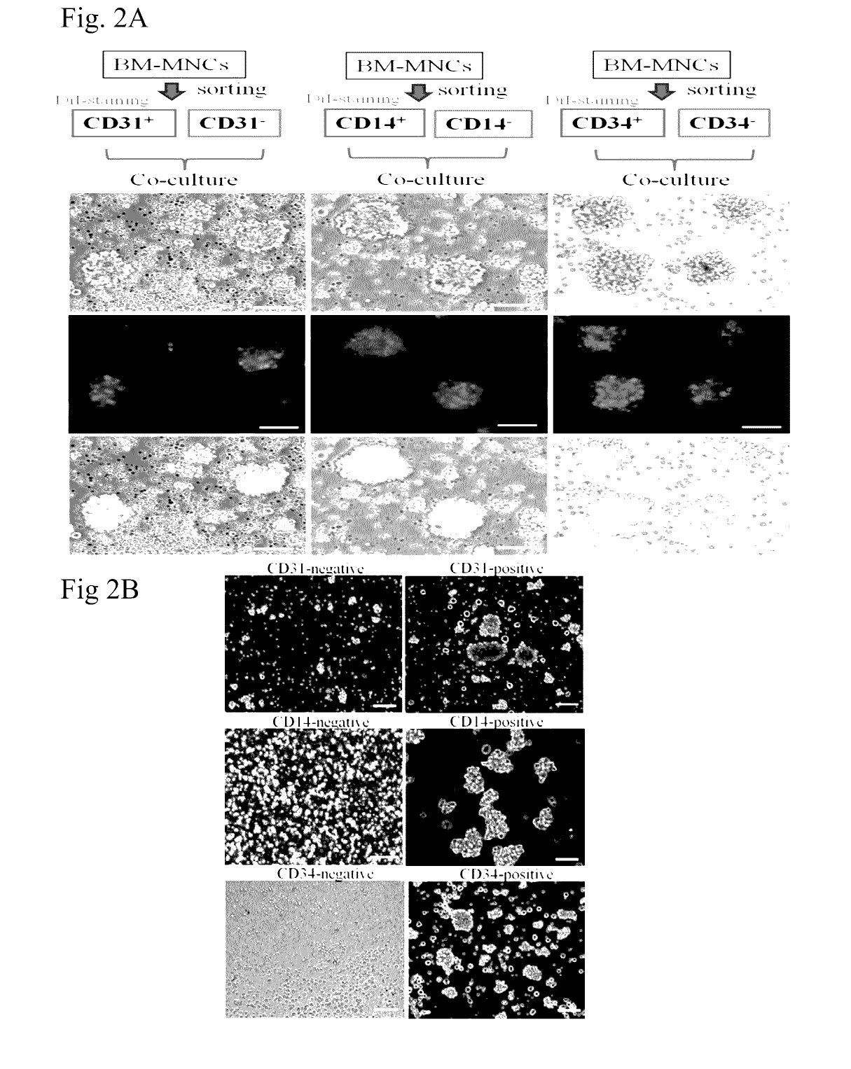

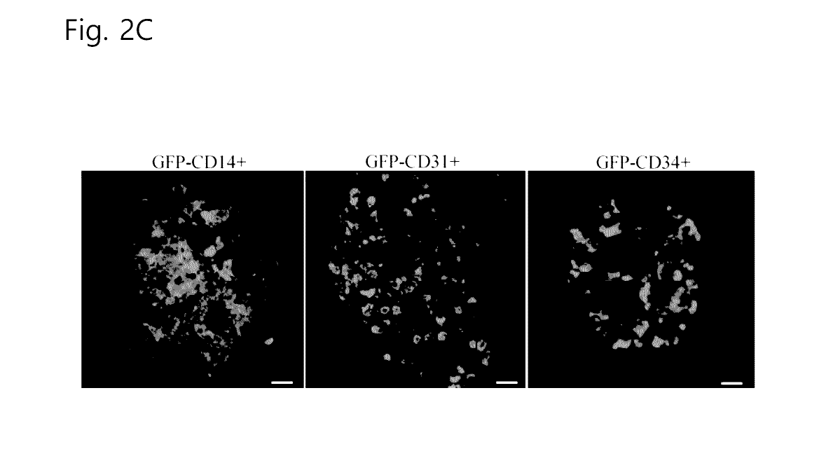

As a result, a positive population was stained with DiI and a negative population was not stained and then co-cultured as shown in FIG. 2. In this case, it was confirmed that only the cells stained with DiI constituted a spheroid (FIG. 2A). When the populations were separately cultured, the positive population formed a spheroid but the negative population could not form a spheroid (FIG. 2B).

Further, only a positive population of each marker in a GFP-Tg mouse was sorted and a negative population of each marker in a wild-type mouse was sorted and then co-cultured. Then, a spheroid was cut so as to observe the inside of the spheroid using a confocal microscope. As a result, it was shown that the inside was formed of positive cells (FIG. 2C).

### Example 3. Confirmation of Angiogenic Capacity of BM-Spheroid In Vitro

The inventors of the present disclosure conducted a matrigel tube formation test to confirm angiogenic capacity of spheroids.

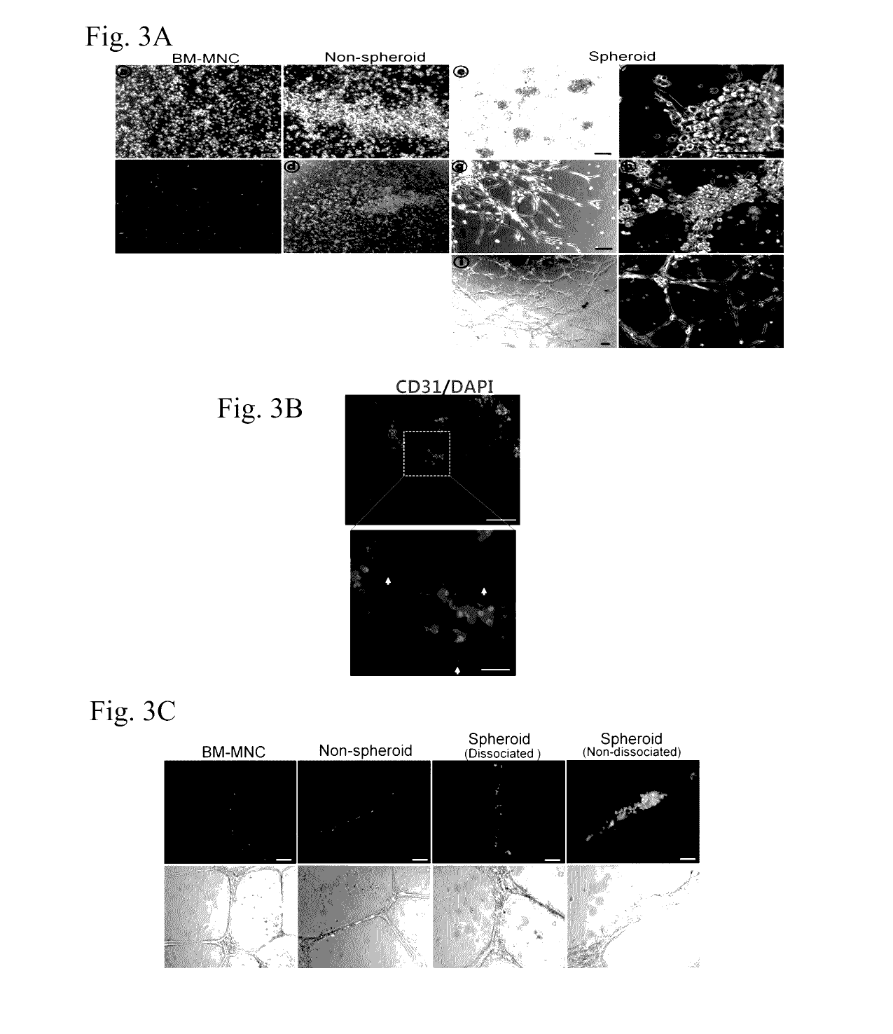

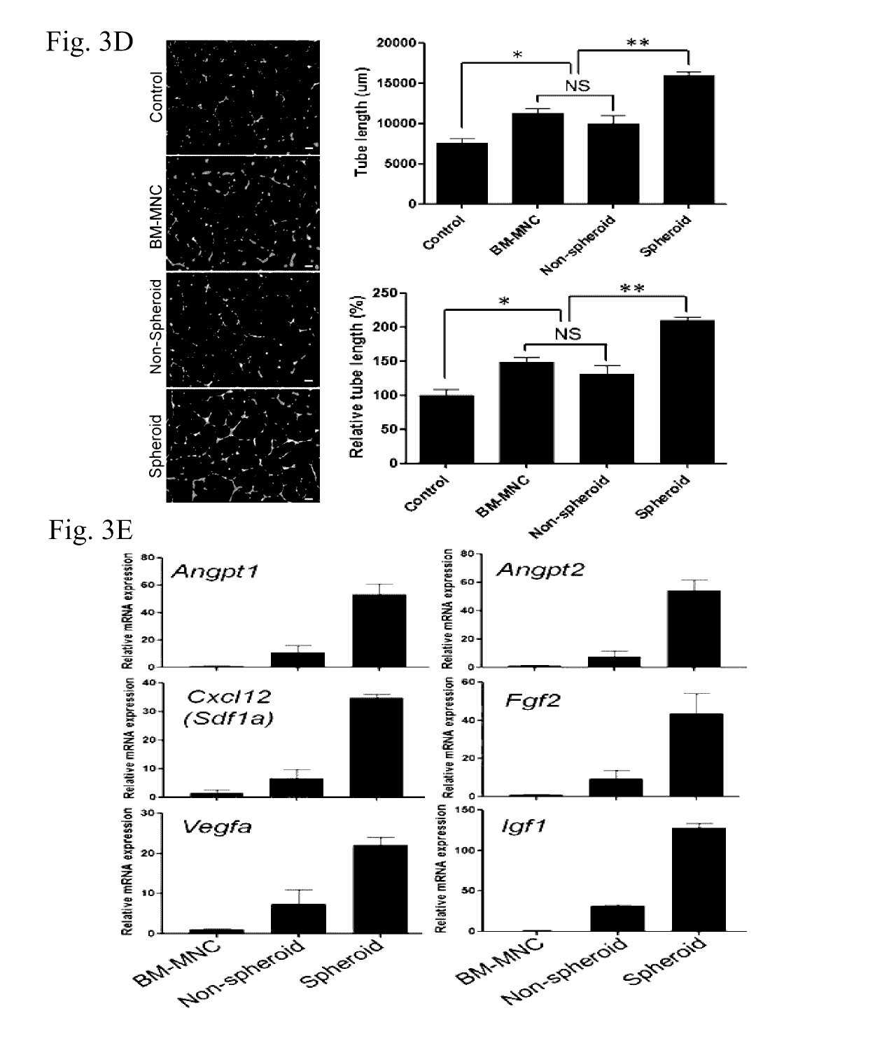

As a result, tubes came out of the spheroids and were connected to each other to form a network as shown in FIG. 3 (FIG. 3A), and these tubes were positive to immunostaining with CD31 as an antibody to an endothelial cell (FIG. 3B). Meanwhile, BM-non-spheroids and fresh bone marrow mononuclear cells could not form a blood vessel structure.

Further, it was observed that GFP-BM-spheroids derived from a GFP-Tg mouse incorporated into a blood vessel structure formed by HUVEC and more GFP-BM-spheroids incorporated into the blood vessel than the fresh bone marrow mononuclear cells or BM-non-spheroids (FIG. 3C).

Meanwhile, in order to evaluate the effects on factors secreted from the BM-spheroids, a culture membrane insert was placed on an upper end within a culture dish to isolate HUVEC on a matrigel and the spheroids were put into the culture membrane insert and then kept for 24 hours.

As a result, it was observed that the BM-spheroids formed more blood vessels than the fresh bone marrow mononuclear cells or the BM-non-spheroids (FIG. 3D).

Further, it was observed that the BM-spheroids had higher expression levels of the factors involved in proliferation of blood vessels than the freshly isolated bone marrow mononuclear cells or the BM-non-spheroids (FIG. 3E), and these factors showed that factors secreted from spheroids in vitro may cause improvement in matrigel tube formation of endothelial cells.

### Example 4. Confirmation of Angiogenic Capacity of BM-Spheroid in Matrigel Plug In Vivo

The inventors of the present disclosure carried out a matrigel plug assay in order to evaluate whether BM-spheroids have angiogenic capacity in vivo, and performed immunofluorescent staining with CD31 antibody to a vascular density within a matrigel plug on the 14^{th} day.

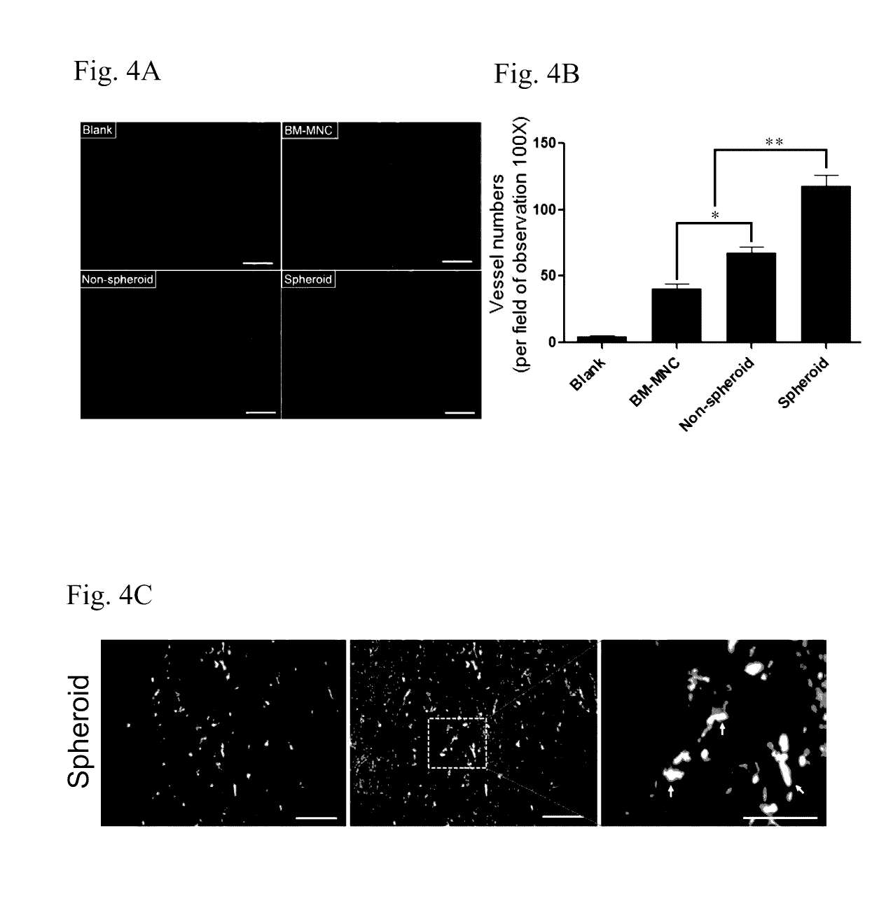

As a result, a group injected with BM-spheroids showed a remarkable increase of new blood vessels by comparison with a blank, a BM-MNC, and a BM-non-spheroid as shown in FIG. 4 (FIGS. 4A and 4B).

Further, it was observed that GFP-BM-spheroids were located around blood vessels stained with CD31 or directly incorporated into the blood vessels (FIG. 4C).

These results show that BM-spheroids can contribute to formation of new blood vessels in vivo.

### Example 5. Confirmation of Improvement in Blood Sugar Regulation Capacity by Co-Transplantation of BM-Spheroid and Islet Cluster

The inventors of the present disclosure examined the result of co-transplantation of an islet cluster and a BM-spheroid with angiogenic capacity into syngeneic diabetes mouse models.

Firstly, before islet transplantation, Calcein AM and PI were used to evaluate the viability of BM-spheroids and BM-non-spheroids, and it was confirmed that the viability of the BM-spheroids and the BM-non-spheroids was 90% or more.

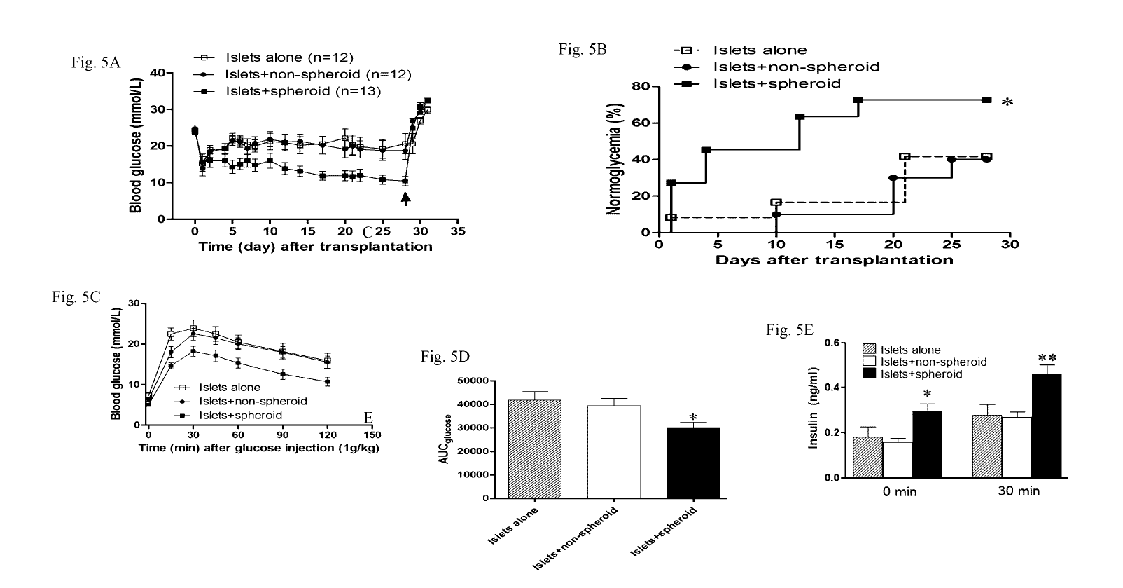

As a result, as shown in FIGS. 5A and 5B, a mean blood sugar level of an islet cluster+spheroid group was significantly lower than that of an islet-alone group and that of an islet cluster+non-spheroid group (FIG. 5A) and the islet cluster+spheroid group also had a higher cumulative percentage of normal blood sugar level arrival with time (FIG. 5B).

Further, in order to examine the function of islet cells transplanted in vivo, intraperitoneal glucose tolerance tests (IPGTT) were conducted on the 28^{th} day after transplantation.

As a result, as shown in FIGS. 5C and 5D, the islet cluster+spheroid group showed an improved change in blood sugar level after glucose tolerance tests (FIG. 5C) and the islet cluster+spheroid group had a significant lower value of the area under the glucose curve (AUGglu) in the glucose tolerance tests, compared with the islet-alone group and the islet cluster+non-spheroid group (FIG. 5D).

Meanwhile, it was confirmed whether or not the improved blood sugar regulation in the islet cluster+spheroid group resulted from increased insulin production from the transplanted islet cells. Further, the islet cluster+spheroid group had a higher concentration of mouse insulin in serum than the islet-alone group and the islet cluster+non-spheroid group (FIG. 5E).

### Example 6. Confirmation of Improvement in Angiogenesis and Proliferation of Beta Cell by Co-Transplantation of BM-Spheroid and Islet Cluster

The inventors of the present disclosure extracted the kidney transplanted on the 14^{th} day and on the 28^{th} day in Example 5 and performed immunofluorescent staining with CD31 antibody in order to evaluate the degree of angiogenesis.

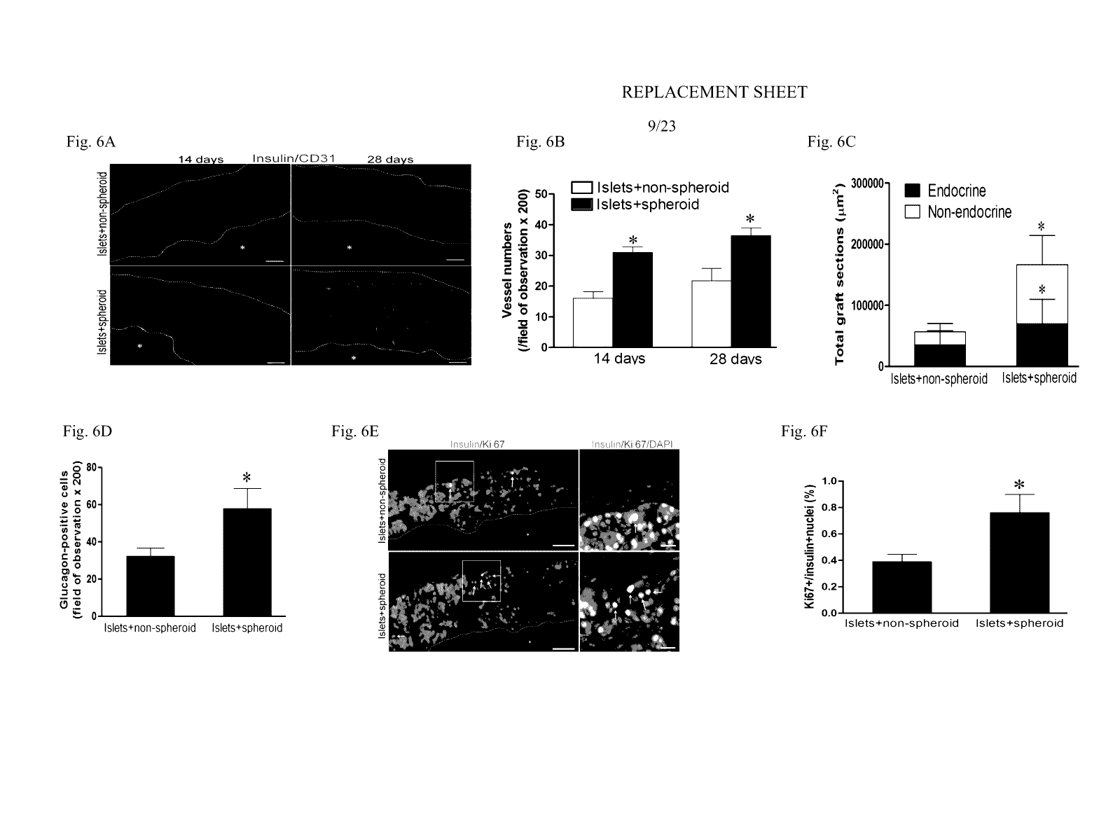

As a result, as shown in FIG. 6, a BM-spheroid and islet cluster co-transplanted group had a remarkably higher vascular density (FIG. 6A) and blood vessels were observed in or around the endocrine zone (FIG. 6B).

Further, the islet cluster+spheroid group was significantly wider in the endocrine zone and the non-endocrine zone than the islet cluster+non-spheroid group (FIG. 6C). In the BM-spheroid and islet cluster co-transplanted group, most of glucagon-positive alpha cells were distributed around the endocrine zone, whereas in the islet cluster+non-spheroid group, alpha cells were irregularly distributed.

Furthermore, the two groups showed a significant difference in number of alpha cells (FIG. 6D), and the cluster co-transplanted group showed improved proliferation of beta cells (FIGS. 6E and 6F).

These results suggest that co-transplantation of a BM-spheroid and an islet cluster improves proliferation of beta cells.

### Example 7. Involvement of BM-Spheroid in Angiogenesis

The inventors of the present disclosure checked whether or not a blood vessel formed by injecting TRITC-conjugated BS1-lectin into a GFP-Tg donor mouse was stained in order to evaluate functional blood vessels in the transplanted tissue and the degree of contribution of functional blood vessels derived from the donor.

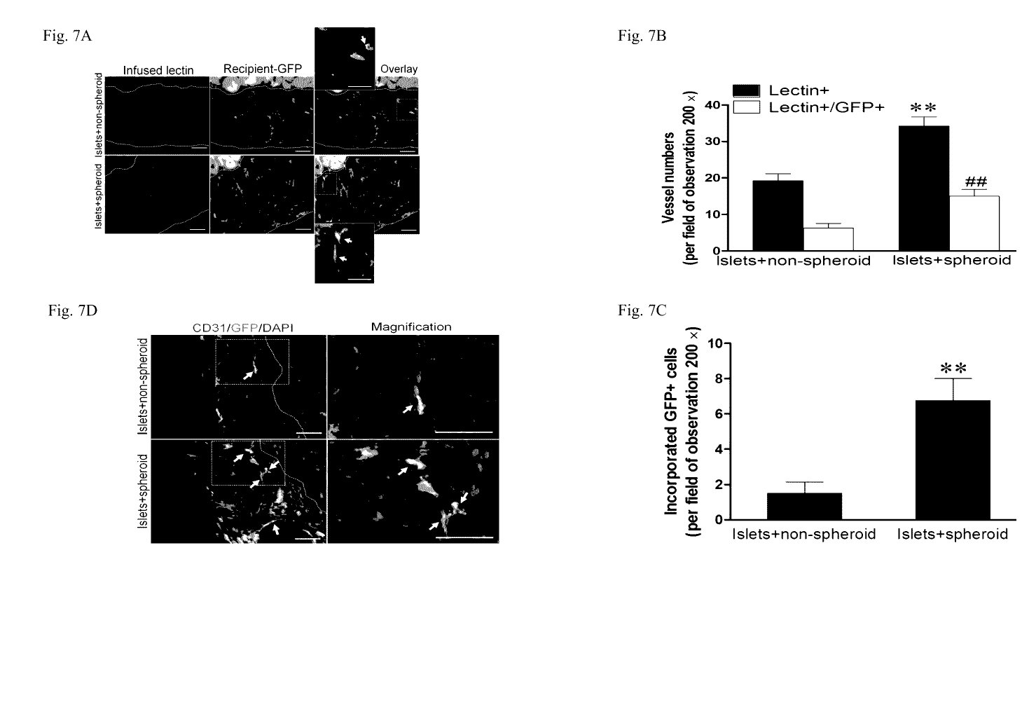

As a result, as shown in FIG. 7, significantly more positive blood vessels stained with the injected lectin were observed in a BM-spheroid group than in a BM-non-spheroid group (FIGS. 7A and 7B). Further, an islet cluster+BM-spheroid group had a remarkably higher density of functional blood vessels derived from the donor than an islet cluster+non-spheroid group, and it was observed that the transplanted GFP-spheroid was distributed in or around the endocrine zone and more cells of a spheroid co-transplanted group were stuck in blood vessels (FIGS. 7C and 7D).

### Example 8. Co-Transplantation of Spheroid and Islet Cluster Through Hepatic Portal Vein

The inventors of the present disclosure performed transplantation through the hepatic portal vein in order to confirm the effects of co-transplantation of a spheroid and an islet cluster through the hepatic portal vein.

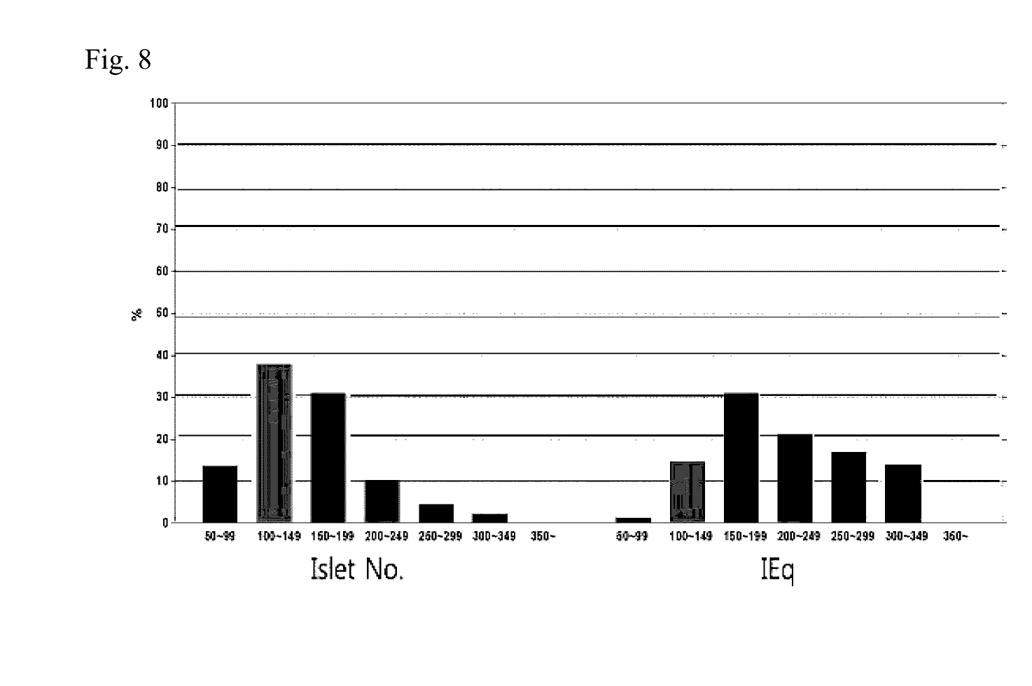

FIG. 8 shows the composition of human islet cells according to size. The human islet cells generally have a size of 50 to 350 μm, and most of islets have a size of 100 to 200 μm. Since the cells are transplanted through the hepatic portal vein, if they are too large, blood vessels are blocked and embolism occurs. Therefore, it is important to prepare BM-spheroids to a size similar to that of human islet cells to be transplanted.

The left graph shows distribution of actual number of islets according to size of isolated islet cells. However, even if the number of small islets is high, the volume actually occupied by the islets is relatively small (in proportion to insulin secretion capacity). That is, the number of islet cells can be applied regardless of cell size, and, thus, even if an equal number of cells are transplanted, a function or result may be different from each other. Therefore, islet cells in various sizes are converted and newly counted as islet equivalents, i.e., IEq (right graph) on the basis of an islet diameter of 150 μm. Therefore, islet cells can be defined as a value converted on the basis of an islet size of 150 μm regardless of a size of the isolated islet. In the left and right graphs, islets having a size of 50 to 99 μm are large in number but insignificant in number on the basis of 150 μm.

That is, it can be seen that islets having a size of 100 to 350 μm are important.

Further, an islet transplantation site is peculiarly in the liver, and, thus, it is difficult to transplant other cells. If a single cell is transplanted, it is difficult for the cell to stay in the liver due to systemic circulation.

Therefore, the inventors of the present disclosure prepared spheroids to a size similar to that of an islet cluster and transplanted them in order for the spheroids to stay in the liver for a longer time and thus improve the function of islet cells.

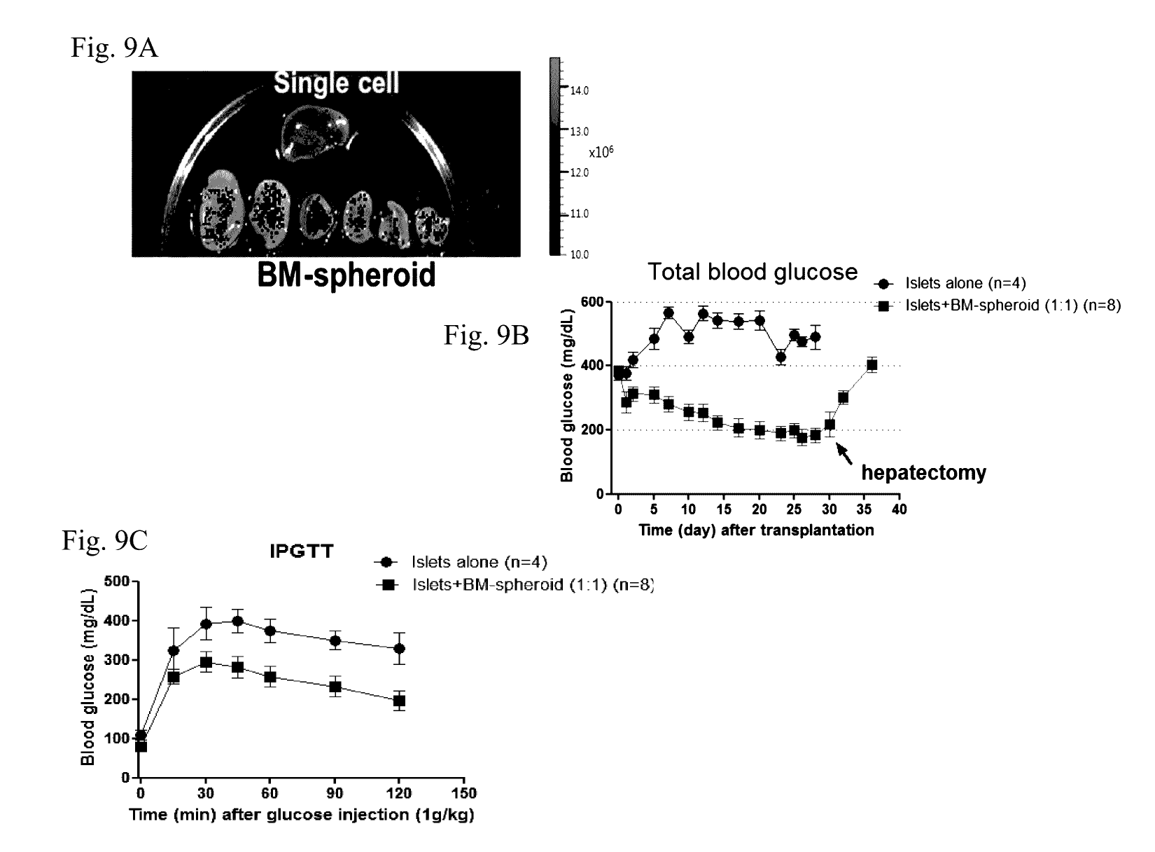

As a result, as shown in FIGS. 9A-9C, a group in which BM-spheroids having a size similar to an islet cluster and the islet cluster were co-transplanted showed a remarkably improved blood sugar regulation capacity, compared with an islet-alone group. FIG. 9A shows a photograph of a single cell and BM-spheroids. FIG. 9B is a chart of total blood glucose levels versus time after transplantation. FIG. 9C is a chart of total blood glucose levels versus time after glucose injection (IPGTT).

### Example 9. Comparison with MSC-Spheroid

The inventors of the present disclosure prepared mesenchymal stem cell (MSC)-spheroids by a hanging-drop method and compared them in order to confirm improvement in engraftment and viability of islet cells in case of co-transplantation of a BM-spheroid and an islet cluster of the present disclosure through the hepatic portal vein.

In short, to prepare MSC-spheroids, each drop was arranged inside the lid of a dish according to the number of mesenchymal stem cells (1, 2.5, 5, 10×10⁴) per 20 μl and the lid was turned over to cover the dish, so that the cells were cultured for 3 days and then spheroids were obtained.

In case of transplanting the prepared MSC-spheroids through the hepatic portal vein, it was checked whether or not a pathological phenomenon occurred.

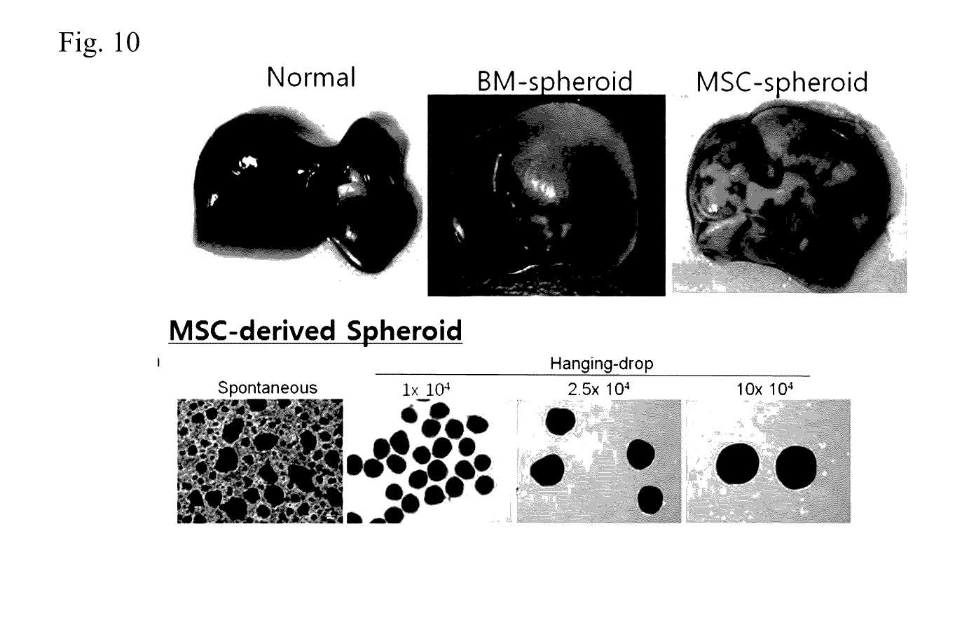

As a result, as shown in FIG. 10A, in case of injecting the BM-spheroids of the present disclosure, any pathological phenomenon (thrombosis or embolism) was not observed, whereas in case of the MSC-spheroids, a pathological phenomenon such as necrosis was clearly observed from a liver surface.

### Example 10. Comparison Depending on Degree of Integration of Spheroid

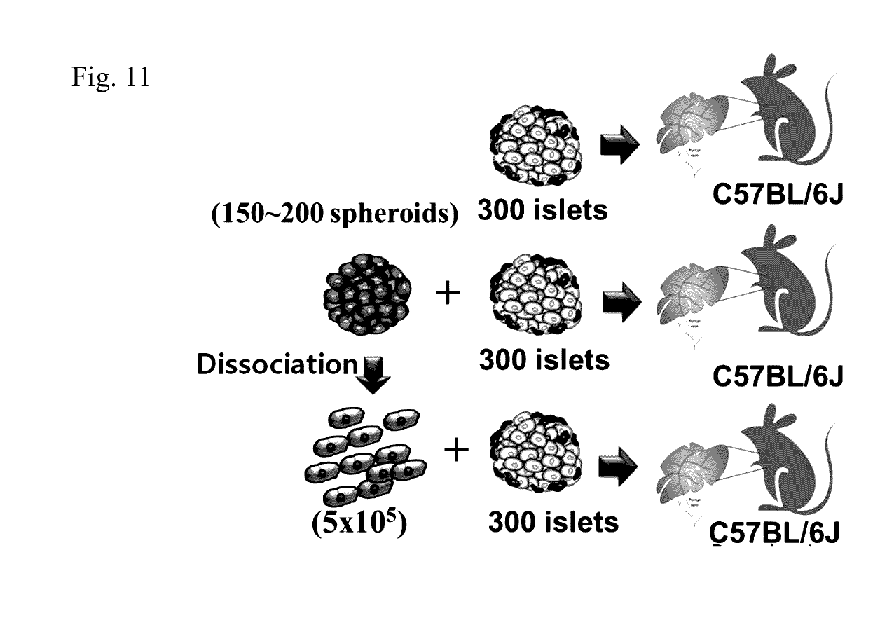

The inventors of the present disclosure prepared BM-spheroid-dissociated cells in which clustered BM-spheroids of the present disclosure were dissociated and compared them in order to confirm improvement in engraftment and viability of islet cells depending on the degree of integration of spheroids in case of co-transplantation of a BM-spheroid and an islet cluster of the present disclosure through the hepatic portal vein (see FIG. 11).

10-1. Comparison about Blood Sugar Level

In order to examine blood sugar regulation capacity, transplantation of an islet cluster alone, co-transplantation of a BM-spheroid and an islet cluster of the present disclosure, and co-transplantation of a BM-spheroid-dissociated cell and an islet cluster were respectively performed to streptozotocin-induced diabetic mice through the hepatic portal vein and then glucose concentration in blood was checked.

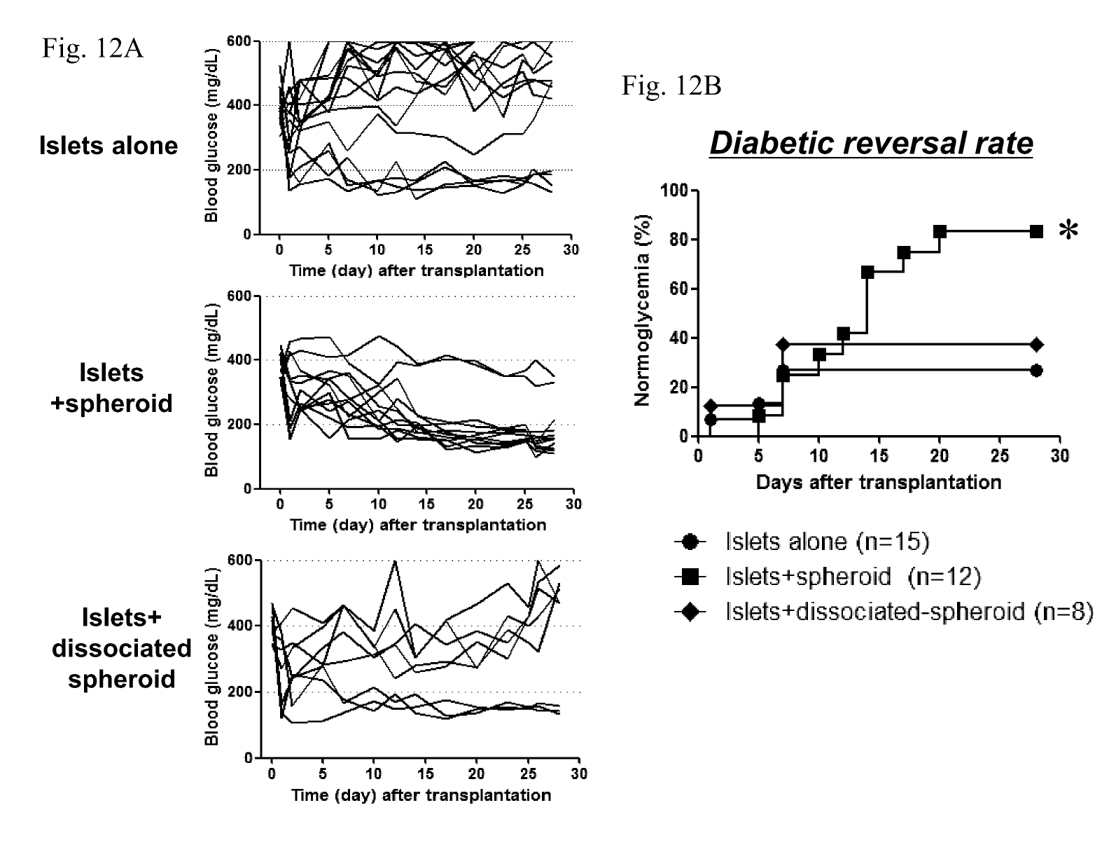

As a result, as shown in FIG. 12A and FIG. 12B, in case of blood sugar change for 28 days, the co-transplantation of the BM-spheroid and the islet cluster of the present disclosure through the hepatic portal vein showed improved blood sugar regulation capacity (FIG. 12A) and improved diabetes cure rate, compared with the other controls.

### 10-2. Confirmation of Function of Transplanted Islet Cell

In order to examine a function of islet cells transplanted on the 28^{th} day, transplantation of an islet cluster alone, co-transplantation of a BM-spheroid and an islet cluster of the present disclosure, and co-transplantation of a BM-spheroid-dissociated cell and an islet cluster were respectively performed to streptozotocin-induced diabetic mice through the hepatic portal vein and then glucose tolerance and fasting glucose concentration in blood were checked.

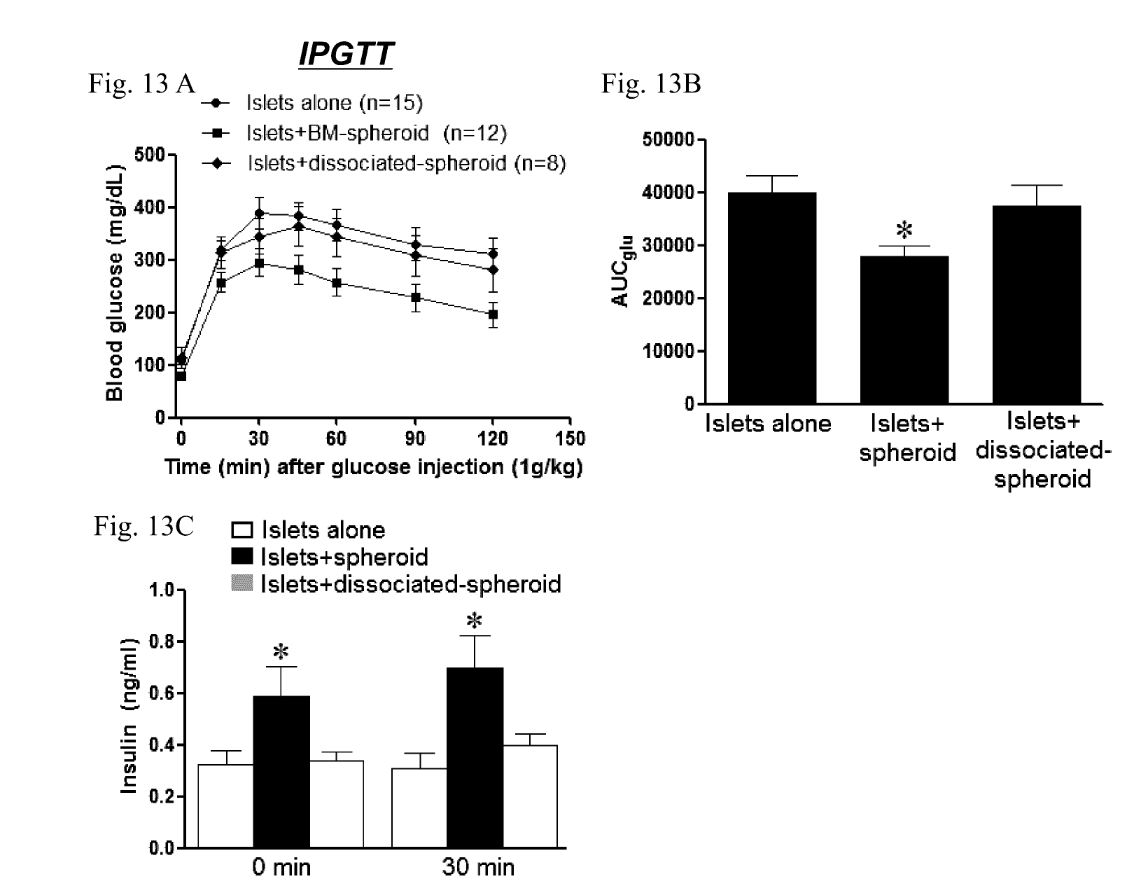

As a result, as shown in FIG. 13A to FIG. 13C, in case of checking a blood sugar change with time after peritoneal injection of glucose (1 g/kg), it was confirmed that the BM-spheroid and islet cluster co-transplanted group transplanted through the hepatic portal vein according to the present disclosure showed rapidly regulated a blood sugar level (FIG. 13A and FIG. 13B), compared with the other controls. Further, in case of checking insulin concentration in blood at 0 and 30 minutes, it was confirmed that the BM-spheroid and islet cluster co-transplanted group transplanted through the hepatic portal vein according to the present disclosure secreted the highest level of insulin, compared with the other controls (FIG. 13C).

### 10-3. Morphological Confirmation of Transplanted Islet Cell

In order to examine morphology of islet cells transplanted through the hepatic portal vein, transplantation of an islet cluster alone, co-transplantation of a BM-spheroid and an islet cluster of the present disclosure, and co-transplantation of a BM-spheroid-dissociated cell and an islet cluster were respectively performed to streptozotocin-induced diabetic mice through the hepatic portal vein and then morphology of the islet cells transplanted into the liver, a ratio of cells, a size of the islet cells, and the number of the islet cells were checked.

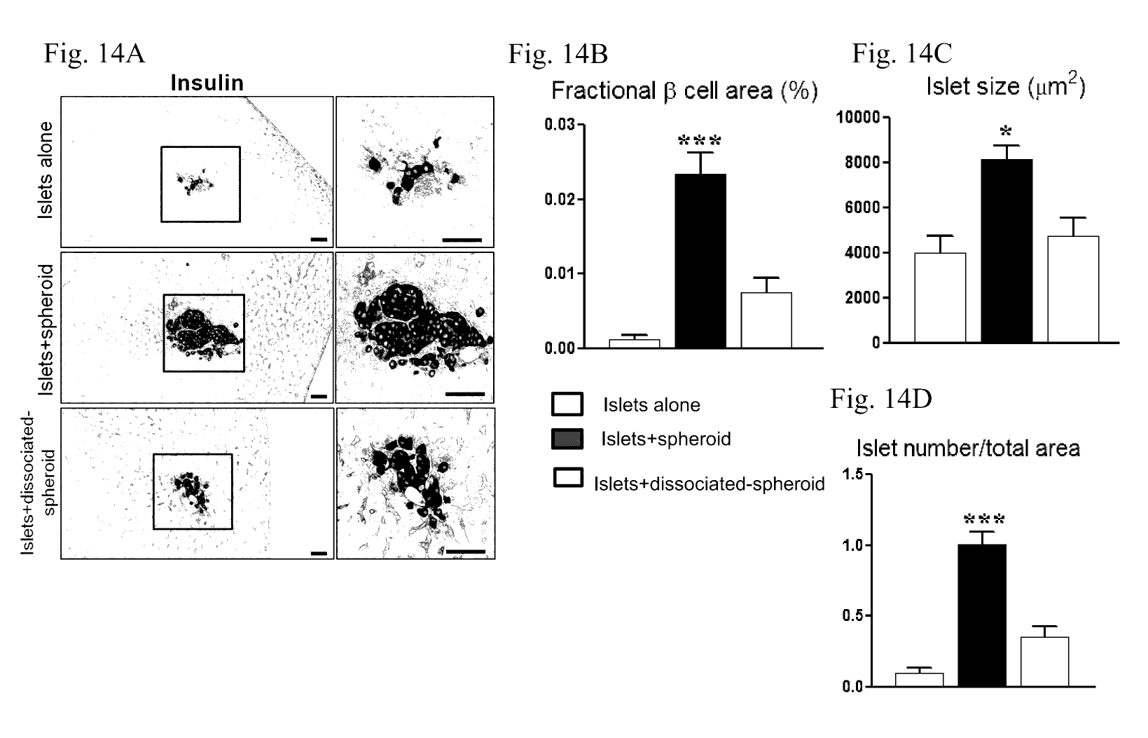

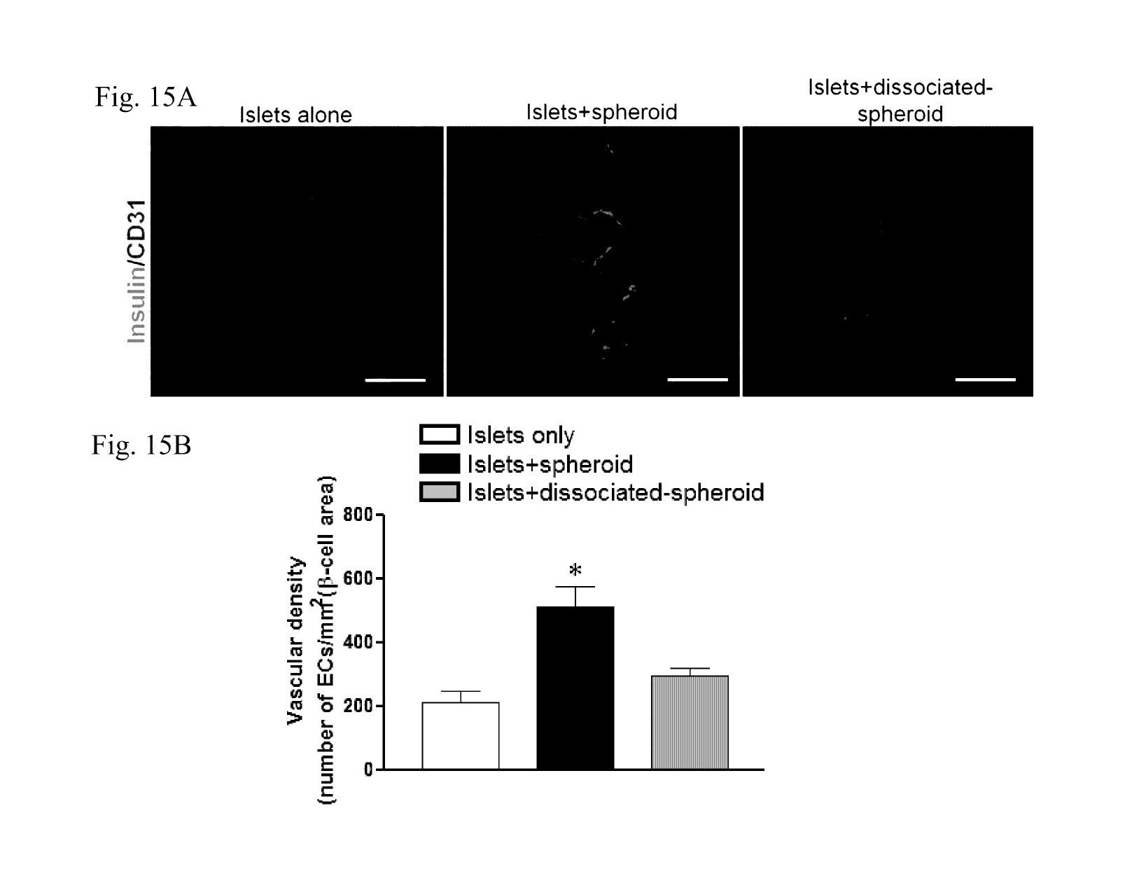

As a result, as shown in FIG. 14A and FIG. 14B, in the BM-spheroid and islet cluster co-transplanted group transplanted through the hepatic portal vein according to the present disclosure compared with the other controls, the islet cells transplanted into the liver maintained intact morphology (FIG. 14A) and a ratio of (3-cells (FIG. 14B), a size of the islet cells (FIG. 14C), and the number of the islet cells (FIG. 14D) were significantly high.

### 10-4. Confirmation of Angiogenic Capacity of Transplanted Islet Cell

In order to evaluate angiogenic capacity of islet cells transplanted through the hepatic portal vein, transplantation of an islet cluster alone, co-transplantation of a BM-spheroid and an islet cluster of the present disclosure, and co-transplantation of a BM-spheroid-dissociated cell and an islet cluster were respectively performed to streptozotocin-induced diabetic mice through the hepatic portal vein and then the degree of angiogenesis was checked using fluorescence images.

As a result, as shown in FIG. 15A and FIG. 15B, improved angiogenesis was observed from the BM-spheroid and islet cluster co-transplanted group transplanted through the hepatic portal vein according to the present disclosure compared with the other controls. This result was as shown in the graph of FIG. 15B.

### 10-5. Tracking of Transplanted BM-Spheroid

In order to track BM-spheroids transplanted through the hepatic portal vein, transplantation of an islet cluster alone, co-transplantation of a BM-spheroid and an islet cluster of the present disclosure, and co-transplantation of a BM-spheroid-dissociated cell and an islet cluster were respectively performed to streptozotocin-induced diabetic mice through the hepatic portal vein and then BM-spheroids labelled with Resovist were checked by MRI.

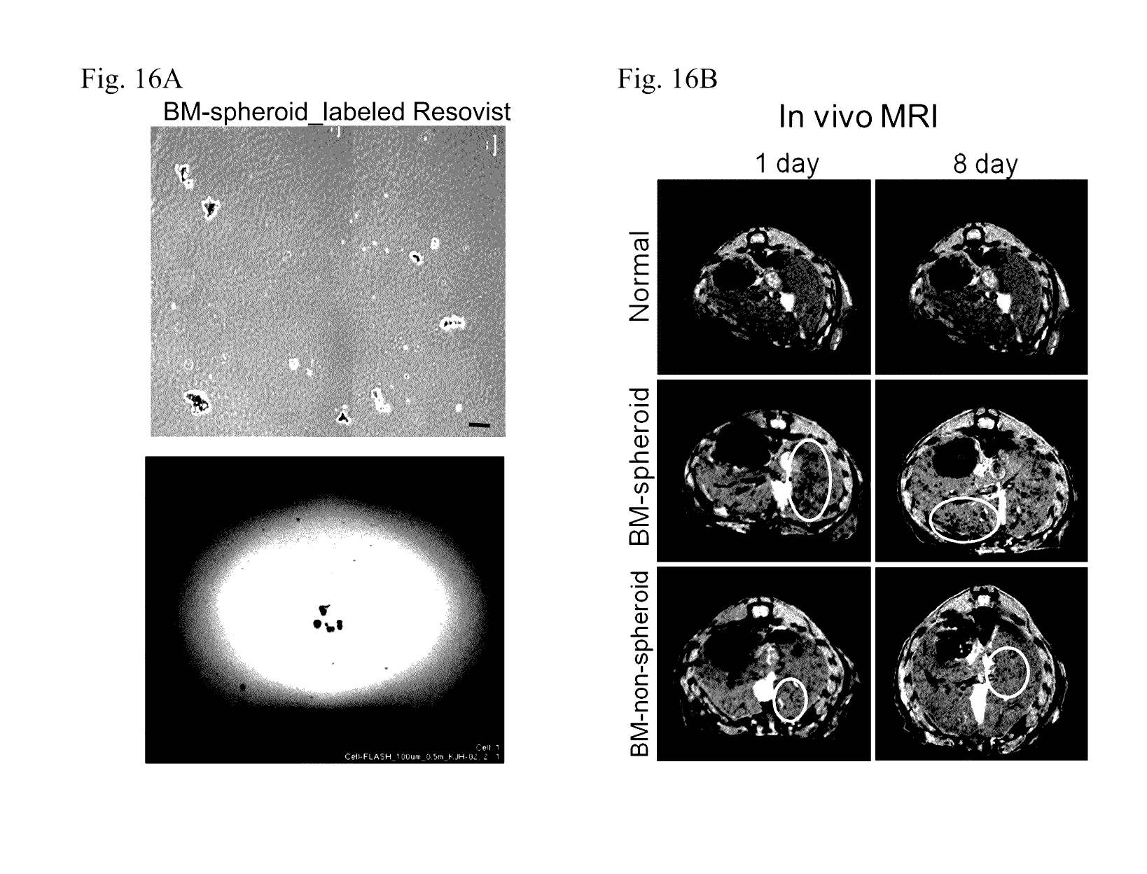

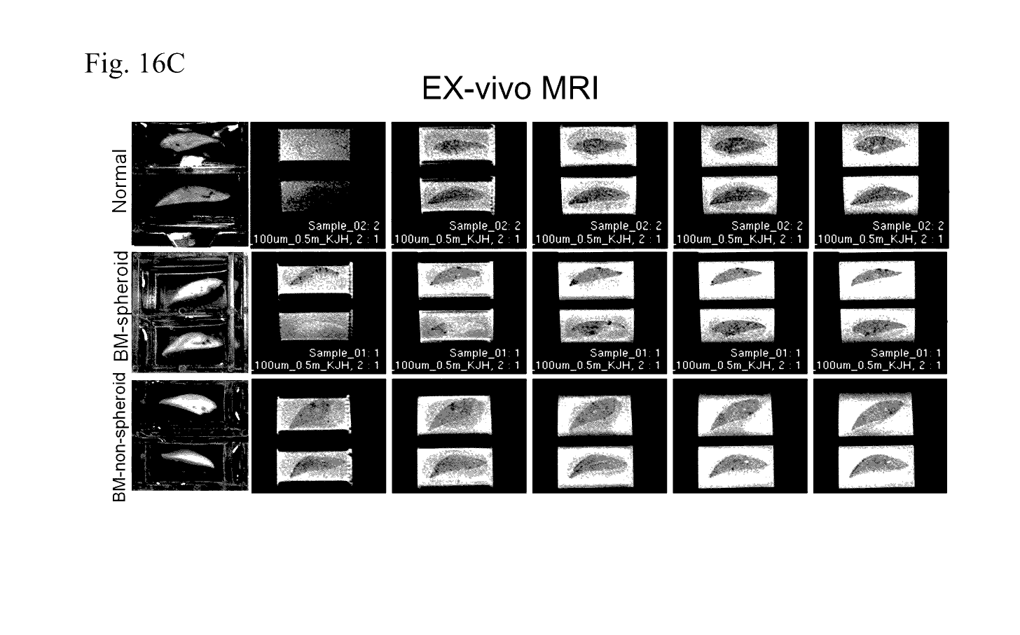

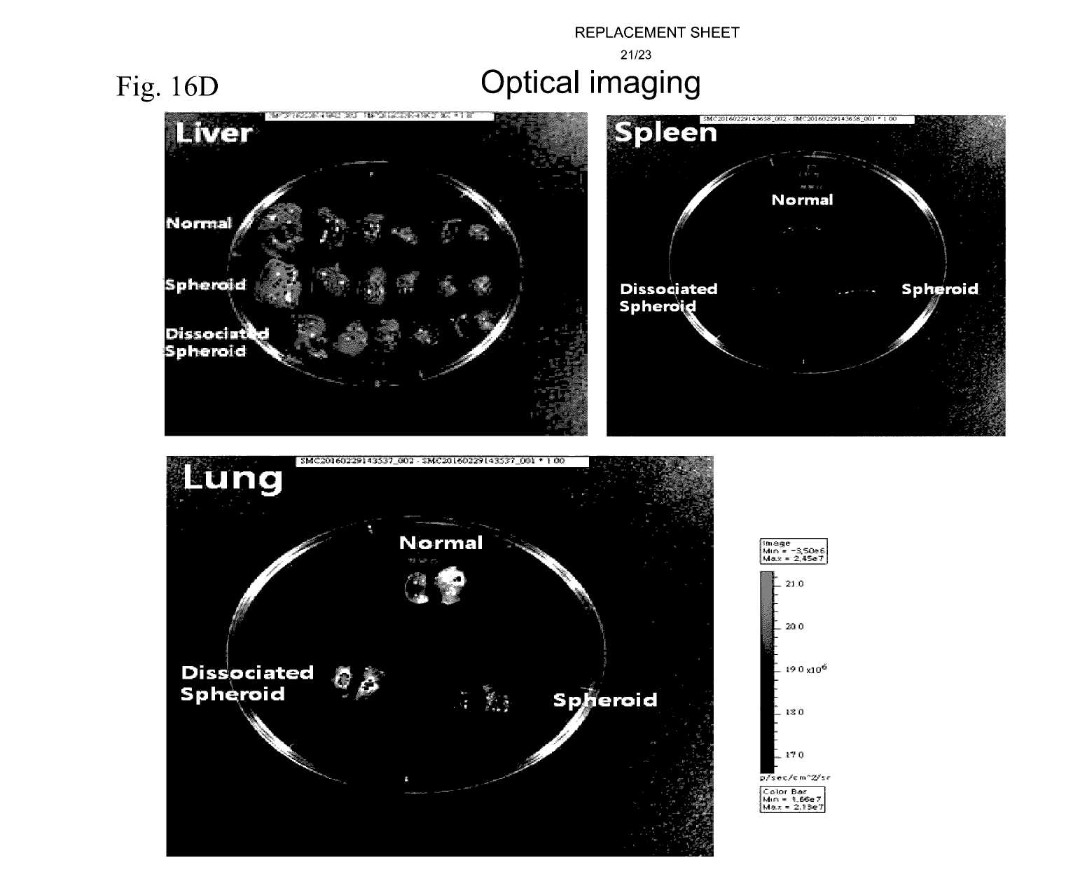

As a result, as shown in FIG. 16A, it was confirmed that BM-spheroids were observed from an in vitro MRI image.

Further, as shown in FIG. 16B, the BM-spheroid and islet cluster co-transplanted group transplanted through the hepatic portal vein and labelled with Resovist according to the present disclosure showed significantly more hypointense spots in an MRI image than the BM-non-spheroid (control) which is a single cell.

Furthermore, as shown in FIG. 16C, the transplanted liver was extracted and MRI-scanned ex vivo, and as a result, the BM-spheroid and islet cluster co-transplanted group transplanted through the hepatic portal vein and labelled with Resovist according to the present disclosure showed significantly more hypointense spots in a liver ex-vivo MRI image than the BM-non-spheroid (control) which is a single cell.

Also, as shown in FIG. 16D, GFP-BM-spheroids prepared from a GFP-mouse were transplanted and on the 8^{th} day, a GFP expression level was checked using an optical imaging device, and as a result, it was confirmed that the BM-spheroid and islet cluster co-transplanted group transplanted through the hepatic portal vein and labelled with Resovist according to the present disclosure was present mostly in the liver, whereas the BM-non-spheroid (control) which is a single cell was present rarely in the liver but present in the lung.

### 10-6. Confirmation of Function of Islet Cell In Vitro Depending on Transplantation Route

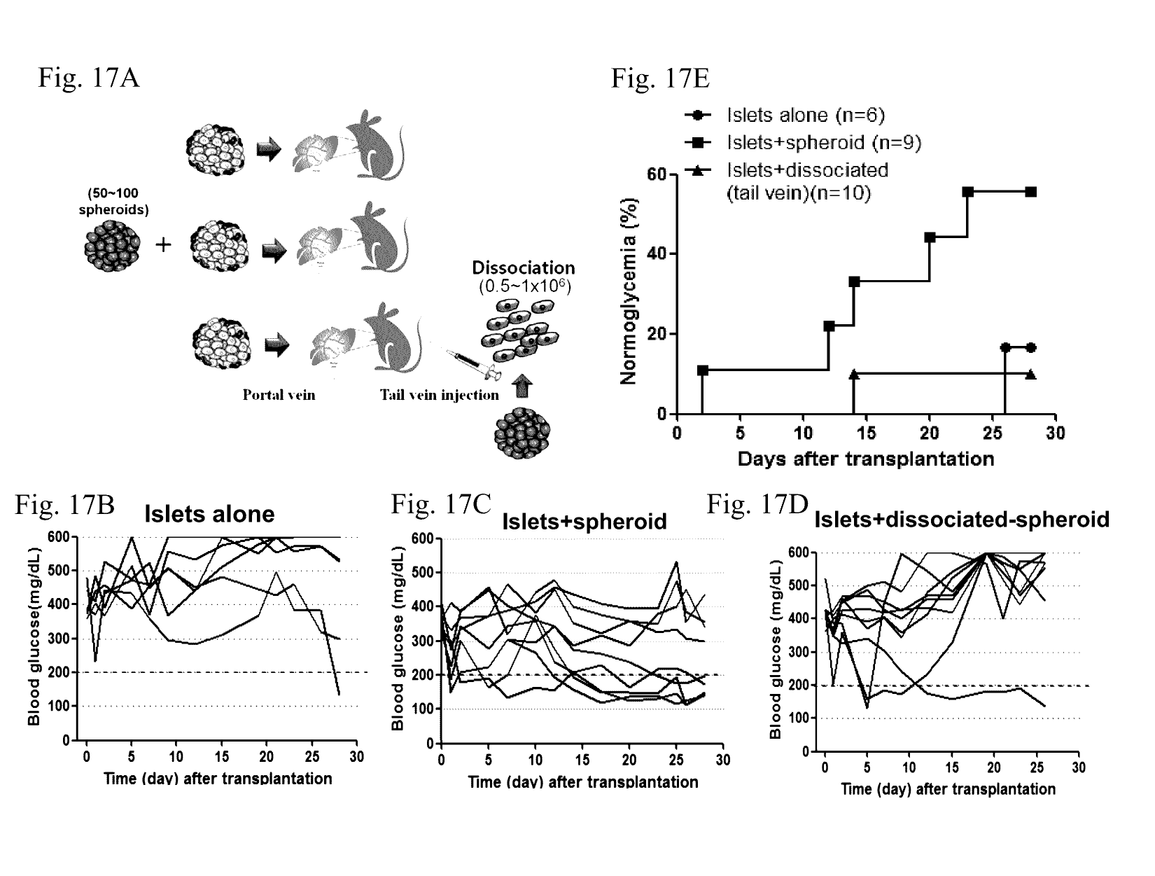

The inventors of the present disclosure evaluated a function of islet cells with respect to co-transplantation through the hepatic portal vein or tail vein. The result thereof is schematically shown in FIG. 17A.

As a result, as shown in FIG. 17E, according to the blood sugar graphs of an islet-alone group (FIG. 17B), a BM-spheroid and islet cluster co-transplanted group transplanted through the hepatic portal vein according to the present disclosure (FIG. 17C), and a group in which islets were transplanted through the hepatic portal vein and then isolated spheroids were transplanted through the tail vein (FIG. 17D), the BM-spheroid and islet cluster co-transplanted group transplanted through the hepatic portal vein according to the present disclosure showed an improved diabetes recovery rate compared with the group transplanted through the tail vein.

### 10-7. Morphological Confirmation of Islet Cell Depending on Transplantation Route

The inventors of the present disclosure checked morphology of islet cells transplanted through the hepatic portal vein or tail vein, a ratio of β-cells, a size of the islet cells, and the number of the islet cells.

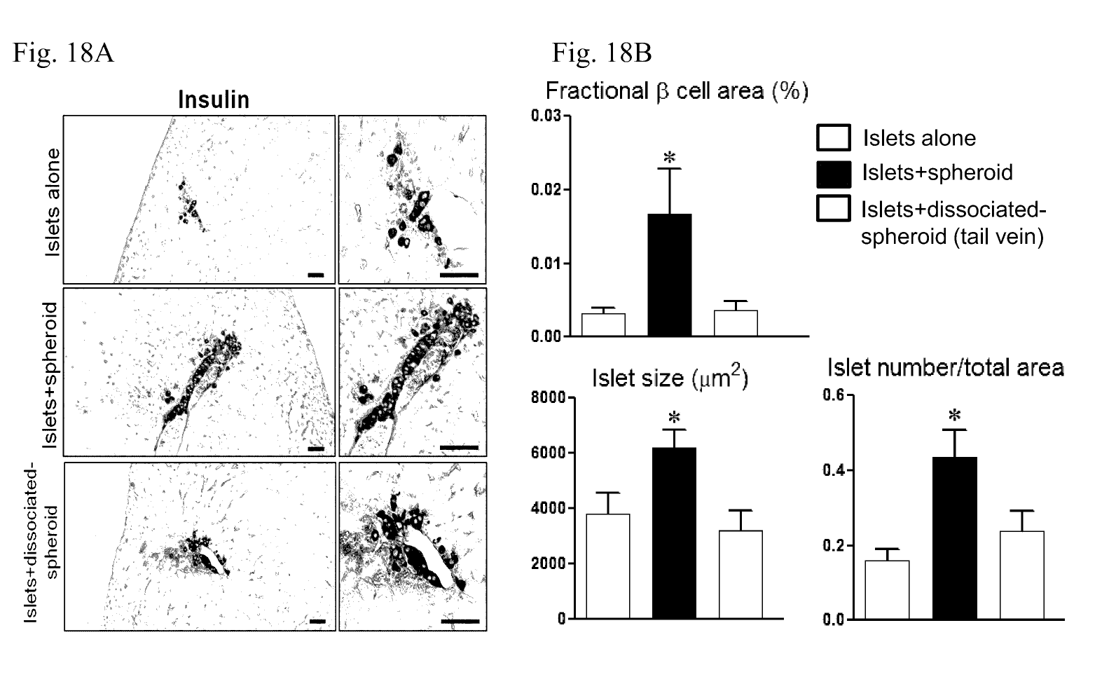

As a result, as shown in FIG. 18A and FIG. 18B, in the BM-spheroid and islet cluster co-transplanted group transplanted through the hepatic portal vein according to the present disclosure compared with the other controls, the transplanted islet cells maintained intact morphology (FIG. 18A) and a ratio of (3-cells (top panel in FIG. 18B), a size of the islet cells (left bottom panel in FIG. 18B), and the number of the islet cells (right bottom panel in FIG. 18B) were significantly high.

While specific parts of the present disclosure have been described in detail, it will be clearly understood by those skilled in the art that the above descriptions are just exemplary embodiments of the present disclosure but do not limit the scope of the present disclosure, which is defined by the accompanying claims and their equivalents.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for treating or preventing diabetes, the method comprising:
administering a composition comprising a spheroid and an islet cluster as active ingredients to a subject in need thereof.

2. The method of claim 1, wherein the diabetes is type 1 diabetes.

3. The method of claim 1, wherein the spheroid is co-transplanted with the islet cluster through the renal capsule or hepatic portal vein.

4. The method of claim 1, wherein a cell mixing ratio of the spheroid and the islet cluster is 1000:1 to 10000:1.

5. The method of claim 1, wherein the spheroid is derived from bone marrow mononuclear cells.

6. The method of claim 1, wherein the spheroid has a diameter of 10 to 500 μm.

7. The method of claim 1, wherein in the spheroid, CD14, CD34, CXCR4, CD14/CXCR4, or CD14/CD34 is overexpressed, as compared with a non-spheroid.

8. The method of claim 1, wherein the spheroid and the islet cluster are derived from autologous, allogenic, or xenogeneic tissues.

9. A method for preparing a cell implant for islet transplantation, the method comprising the following steps:
(a) preparing a spheroid derived from bone marrow mononuclear cells; and
(b) preparing a cell implant by mixing the spheroid and an islet cluster.

10. The method of claim 9, wherein in the cell implant of the step (b), a cell mixing ratio of the spheroid and the islet cluster is 1000:1 to 10000:1.
